(19) United States
(12) Patent Application Publication
(10) Pub. No.: US 2024/0174697 A1
(43) Pub. Date: May 30, 2024

(54) PH-ACTIVABLE FLUORESCENT PROBES FOR TARGETING CELL ORGANELLES

(71) Applicant: Purdue Research Foundation, West Lafayette, IN (US)

(72) Inventors: Gaurav Chopra, West Lafayette, IN (US); Krupal Jethava, West Lafayette, IN (US); Priya Prakash, West Lafayette, IN (US)

(73) Assignee: Purdue Research Foundation, West Lafayette, IN (US)

(21) Appl. No.: 18/281,188

(22) PCT Filed: Mar. 8, 2022
(86) PCT No.: PCT/US22/19223

§ 371 Date: Sep. 8, 2023

Related U.S. Application Data
(60) Provisional application No. 63/160,201, filed on Mar. 12, 2021.

(51) Int. Cl. C07F 5/02; G01N 33/50; G01N 33/58; G01N 33/84

(52) U.S. Cl. CPC C07F 5/022; G01N 33/5079; G01N 33/582; G01N 33/84

(57) ABSTRACT

The present disclosure describes series pH-activable fluorescent probes based on a single BODIPY scaffold selectively targeting lysosomal, mitochondrial, and nucleus. The divergent cell organelle targeting was achieved by synthesizing pH-activable fluorescent probes with differential fluorescence profiles arising due to the presence of a unique functional group in the scaffold. We discovered that the functional group transformation in the same scaffold influences the localization ability of pH-activable fluorescent probes in cell organelle. The development of pH-activable fluorescent probes that target lysosomes and mitochondria organelles in live and fixed primary mouse microglial cells warrants future use in disease-specific biological models.

### CROSS-REFERENCE TO RELATED APPLICATIONS

The present U.S. patent application relates to and claims the priority benefit of U.S. Provisional Patent Application Ser. No. 63/160,201, filed Mar. 12, 2021, the contents of which are hereby incorporated by reference in its entirety into this disclosure.

### TECHNICAL FIELD

The present disclosure generally relates to series pH-activable fluorescent probes based on a single BODIPY scaffold selectively targeting lysosomal, mitochondrial, and nucleus useful for diagnosis and imaging purposes.

### BACKGROUND

This section introduces aspects that may help facilitate a better understanding of the disclosure. Accordingly, these statements are to be read in this light and are not to be understood as admissions about what is or is not prior art.

Fluorescent organic materials have proven to be extremely useful for biological^{1a} and biomedical science.^{1a} Specifically, high-sensitivity fluorescent imaging of cellular organelles with enhanced spatial resolution allows direct visualization of dynamic cellular processes.^{1b} Small-molecule fluorescent probes are essential tools to monitor changes in biological processes in cellular organelles. These include imaging lysosomes,^{2a-b} mitochondria,^{2c} Golgi apparatus,^{2d} nucleus^{2f} among many others, and fluorescent probes are useful to track their abundance, localization, and function in cells. Lysosomes mainly act as a cellular ‘recycling plant’ to maintain intracellular and extracellular homeostasis^{2a} via the breakdown of carbohydrates, lipids, proteins, nucleic acids, cellular debris and other foreign pathogens. Mitochondria is the cellular ‘power plant’ that contains enzymes responsible for energy production needed for biochemical reactions and for energy metabolism to maintain cellular health. In addition, lysosomal and mitochondrial crosstalk is critical for cells and its dysfunction leads to diseases including neurodegeneration.^{2e} Often, imaging of cell organelles, irrespective of pH-activable property, involves specific fluorescent probes having different scaffolds that are prepared separately by multistep synthesis. In that context, a conceptual divergent synthetic strategy delivering a distinct organelle targeting from the same basic scaffold has remained elusive.

Microglia. the immune cells in the brain and macrophages in the periphery phagocytose (or engulf) extracellular material such as bacteria, virus, misfolded proteins, cell debris, etc. from their microenvironment into lysosomes for degradation.^{3a} Lysosomes are membrane-bound acidic organdies containing several enzymes (hydrolases, proteases, lipases, etc.) to actively breakdown the phagocytosed material.^{3b} Microglial cells are an excellent model for examining phagocytosis as well as lysosomal and mitochondrial activity ex vivo and in vivo.^{3c} Microglia are the professional phagocytes in the brain that play a critical role in brain health and development.^{3d} It is known that microglial lysosomes are unable to effectively degrade a large quantity of phagocytosed amyloid-beta aggregates, which may be contributing to neurodegeneration during later stages of Alzheimer's disease.^{3e} Furthermore, during neurodegeneration microglia releases dysfunctional mitochondria into their environment thereby exacerbating neuroinflammation.^{3f}

It is, therefore, crucial to develop pH-activable chemical probes that target lysosomes and mitochondria to understand such cellular processes.^{3c} Fortunately, we can exploit the lysosomal acidic environment of pH 4.5-5.5^{2a,4a} to develop pH-activable fluorescent probes to visualize, track, and investigate lysosomal processes in live and fixed cells and in vivo.^{3c} For targeting mitochondria, we can exploit the negatively charged inner membrane of mitochondria to design a fluorescent probe with a positively charged functional group.^{4b} Importantly, the maintenance of a particular alkaline matrix (pH˜8) by pumping out protons dictates the normal physiological function of mitochondria.^{4c} However, during disease pathogenesis, impaired mitochondria undergo mitophagic elimination through lysosomal fusion.^{2e} Moreover, understanding the crosstalk between mitochondria and lysosomes using targeted fluorescent probes is important for the investigation of cellular processes leading to disease pathogenesis.^{4d} Nonetheless, if mitochondrial targeting fluorescent probe that has acidic pH-activable property, then such fluorescent probes could be useful to track mitochondrial fusion with acidic lysosomes.

### BRIEF DESCRIPTION OF DRAWINGS

FIG. 1 shows our strategy to prepare cell organelle targeting probes in one synthetic scheme with a common intermediate.

FIG. 2 shows Absorption, Fluorescence spectrum of compound LysoShine 1, LysoShine 2, MitoShine at different pH solutions and their pKa values. Spectrum recorded at room temperature in PBS buffer at varying pH with 1% (v/v) of DMSO, in all cases probe concentration=10 μM.

FIGS. 3A-3E. FIG. 3A shows schematic for flow cytometry analysis in live cells. FIGS. 3B-3D demonstrate the fluorescence of LysoShine 1, LysoShine 2, and MitoShine upon uptake by primary mouse microglia (live cells). Modal corresponds to a percentage of the maximum count. FIG. 3E shows the median fluorescence intensity (MFI) values for each probe. US Ctrl is unstained control. Gating strategy and flow plots with commercial dyes in supporting information.

FIG. 4A shows experimental design for fluorescence imaging of primary mouse microglial cells with the (FIG. 4B) LysoShine 1 and (FIG. 4C) LysoShine 2 (green). The localization of the compounds in the lysosomal acidic compartments is shown with the LysoTracker dye (red). Nuclear DNA is stained with DAPI (blue). Scale bars represent 200 μm.

FIG. 5. Fluorescence imaging of primary mouse microglial cells with MitoShine. The localization of the compound was observed in mitochondria (magenta, MitoLite dye) as well as in the acidic lysosomal organelles (red, LysoTracker dye). Insert a on top left corner shows magnified image with nuclear DNA is stained with DAPI (blue). Scale bars represent 50 μm.

FIG. 6 shows a plausible response mechanism of the pH-activable probe.

FIG. 7 describes percent uptake efficiency of the fluorescent probes in BV2 microglia. Cellular uptake of the probe at different concentrations after two hours of incubation. The % uptake efficiency was determined as the percentage of probe taken up by cells out of the total amount of probe in the initial incubation solution. Bars depict n=3 data with SD.

FIG. 8 demonstrates that compound 10 localizes to the nuclei. The compound 10 (green) localizes to the nuclei (blue) in BV2 microglia. Scale bars represent 20 μm.

FIGS. 9A-9B show confocal Imaging of Lysosomal probes LysoShine 1 and LysoShine 2 in BV2 microglia. Confocal images depicting the co-colocalization of (FIG. 9A) LysoShine 1 and (FIG. 9B) LysoShine 2 lysosomal probes (green) with LysoTracker Red DND-99 (LTR, red) in BV2 microglia. Nuclei are stained with DAPI (blue). Scale bars represent 20 μm.

FIG. 10 shows confocal imaging of primary mouse microglial cells with MitoShine. The localization of the compound was observed in mitochondria (magenta) and acidic lysosomal organelles (red). Magnified images on the far-right show MitoShine localization in non-mitochondria (likely lysosomes, FIG. 10a), mitochondria (FIG. 10b), and lysosomes (FIG. 10c). Nuclear DNA is stained with DAPI (blue). Scale bars represent 50 μm.

FIG. 11 shows overlap of lysosomes and mitochondria in primary microglia. The overlap of mitochondria (magenta) and acidic lysosomal organelles (red) observed via confocal microscope during co-treatment with MitoShine florescent probe. Scale bars represent 50 μm.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Unless defined otherwise, all technical and scientific terms used herein have the same meaning as commonly understood to one of ordinary skill in the art. As defined herein, the following terms and phrases shall have the meanings set forth below.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

ABBREVIATIONS: BODIPY, 4,4-difluoro boron dipyrromethane; DMSO, dimethyl sulfoxide; MTT, Microculture Tetrazolium Assay; LDH, Lactate dehydrogenase.

The compounds described herein may contain one or more chiral centers or may otherwise he capable of existing as multiple stereoisomers, it is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to he understood that such mixtures of stereoisomers may include a single stereochemical configuration at one or more chiral centers, while including mixtures of stereochemical configuration at one or more other chiral centers.

Similarly, the compounds described herein may include geometric centers, such as cis, irons, E, and Z double bonds. it is to be understood that in another embodiment, the invention described herein is not limited to any particular geometric isomer requirement, and that the compounds, and compositions, methods, uses, and medicaments that include them may be pure. or may be any of a variety of geometric isomer mixtures. It is also to be understood that such mixtures of geometric isomers may include a single configuration at one or more double bonds, while including mixtures of geometry at one or more other double bonds.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. in cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may he administered via the same or different routes of administration. The compounds or compositions may be administered according to simultaneous or alternating regimens, at the same or different times during the course of the therapy, concurrently in divided or single forms.

The terra “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. in one aspect, the therapeutically effective amount is that which may treat or alleviate the disease or symptoms of the disease at a reasonable bene fittrisk ratio applicable to any medical treatment, However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

In some illustrative embodiments, this disclosure relates to a compound having a formula (I)

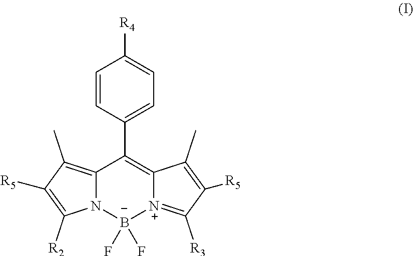

or an acceptable salt thereof, wherein R₂ and R₃, independently, are:

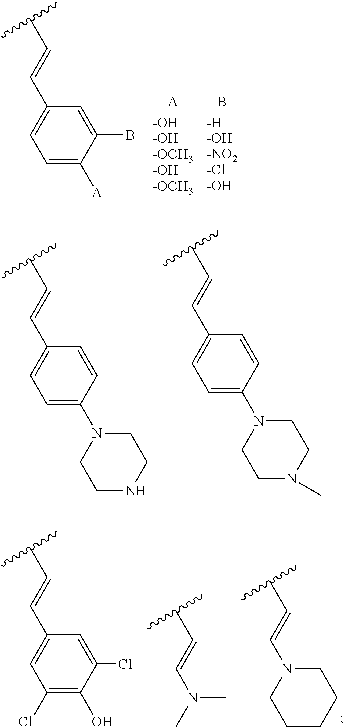

### R₄ is

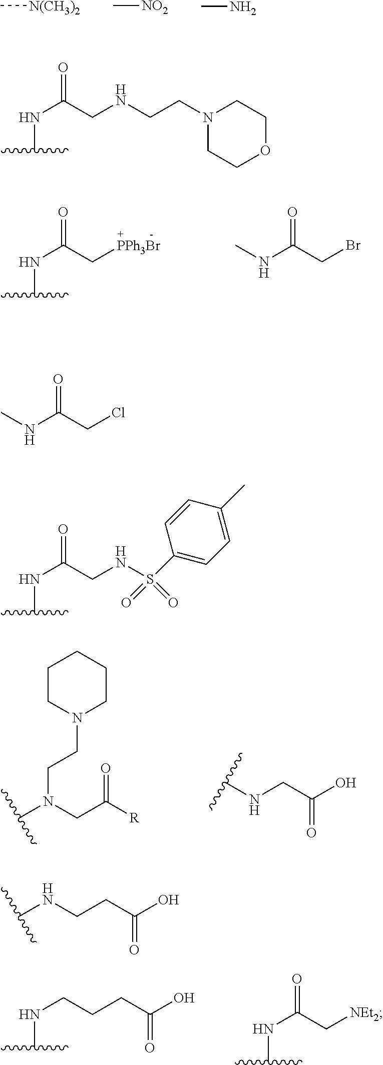 [0043] and

R₅=—CO₂Et, —COOH, —CONHNH₂,

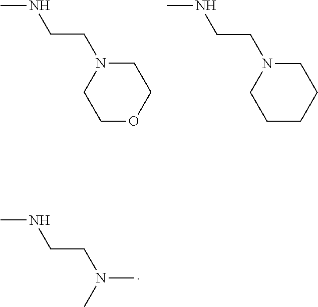

In some illustrative embodiments, this disclosure relates to a compound having a formula (I) as disclosed herein, wherein R₂ and R₃ are methyl; R₄ is —N(CH₃)₂, —NO₂, or —NH₂; and R₅ is —CO₂Et.

In some illustrative embodiments, this disclosure relates to a compound having a formula (I) as disclosed herein, wherein R₅ is —CO₂Et; R₂ and R₃, independently, are:

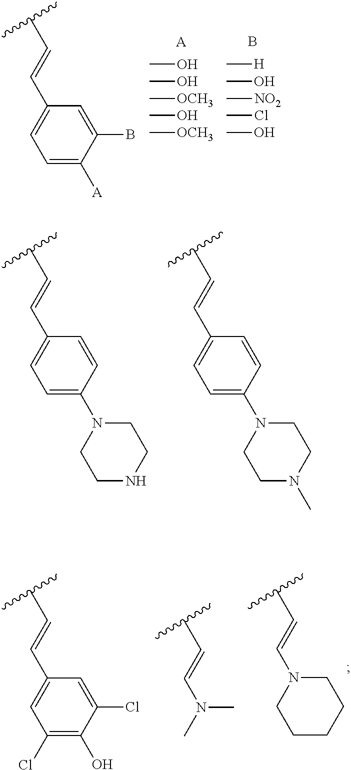

and R₄ is

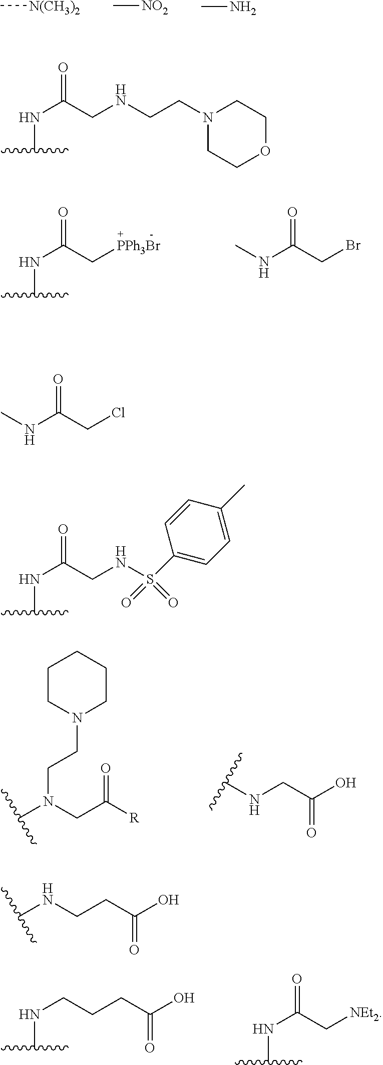

In some illustrative embodiments, this disclosure relates to a compound having a formula (I) as disclosed herein, wherein R₂ and R₃ are methyl; R₄ is

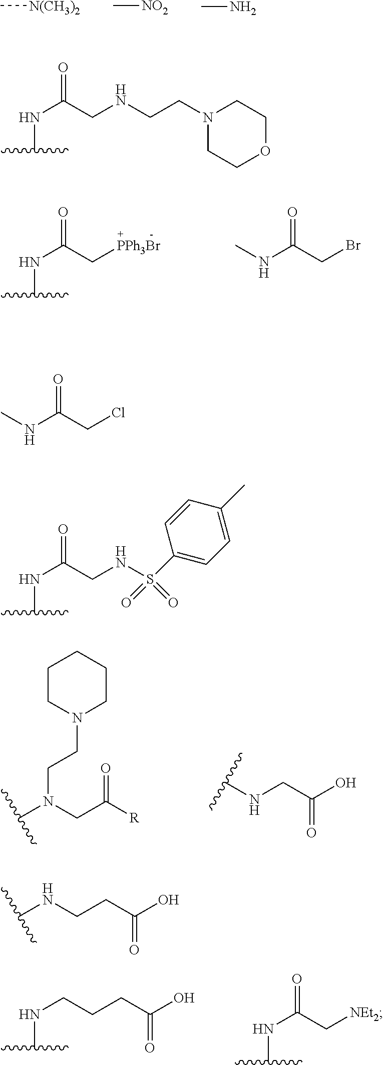

and

R₅=—CO₂Et, —COOH, —CONHNH₂,

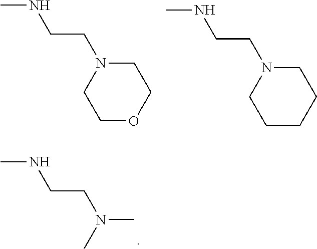

In some illustrative embodiments, this disclosure relates to a compound having a formula (I) as disclosed herein, wherein R₅ is —CO₂Et; R₂ and R₃, independently, are:

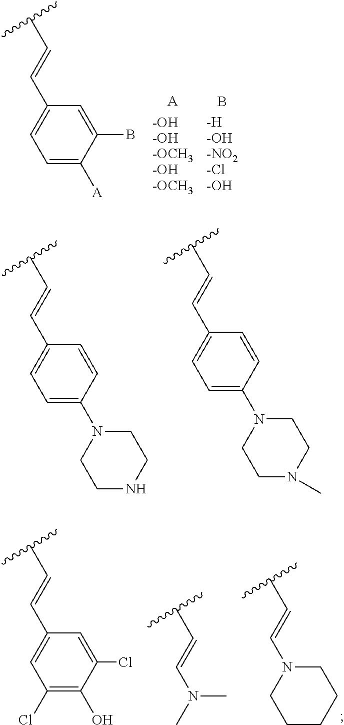

and R₄ is

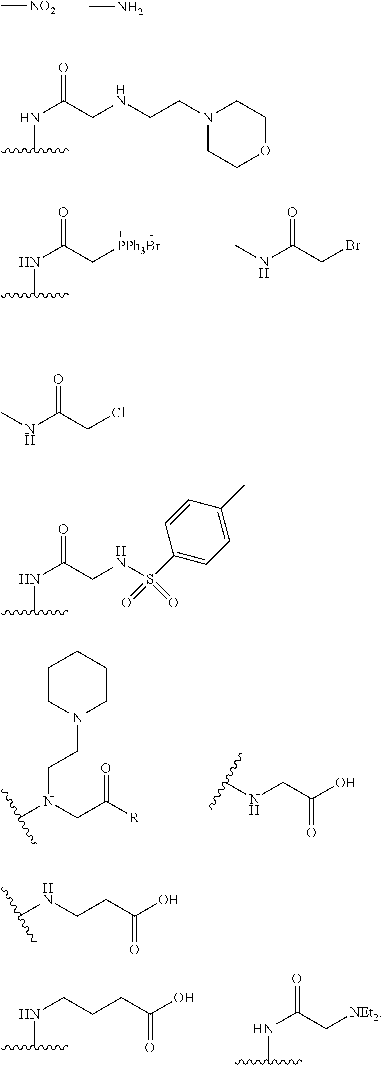

In some illustrative embodiments, this disclosure relates to a compound having a formula (I) as disclosed herein, wherein said compounds are useful for as a lysosomal, mitochondrial, and nucleus targeting pH-activable fluorescent probe.

In some illustrative embodiments, this disclosure relates to a diagnostic kit comprising one or more compounds as disclosed herein.

In some illustrative embodiments, this disclosure relates to a diagnostic kit for imaging comprising one or more compounds as disclosed herein.

In some illustrative embodiments, this disclosure relates to a diagnostic kit comprising one or more compounds as disclosed herein, wherein said compounds target lysosomal, mitochondrial, or nucleus.

In some illustrative embodiments, this disclosure relates to a pH-probe for diagnostic comprising one or more compounds as disclosed herein.

In some illustrative embodiments, this disclosure relates to a pH-probe for imaging comprising one or more compounds as disclosed herein.

In some illustrative embodiments, this disclosure relates to a pH-activable fluorescent probe for diagnostic comprising one or more compounds as disclosed herein.

In some illustrative embodiments, this disclosure relates to a pH-activable fluorescent probe for diagnostic comprising one or more compounds as disclosed herein, wherein said compounds target lysosomal, mitochondrial, or nucleus.

In some illustrative embodiments, this disclosure relates to a diagnostic kit comprising a compound having a formula (I)

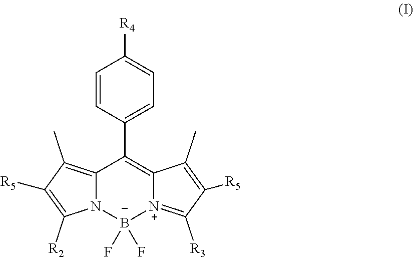

or an acceptable salt thereof, wherein R₂ and R₃, independently, are:

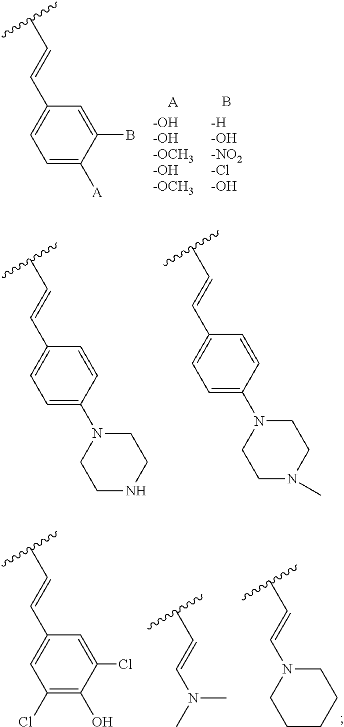

R₄ is

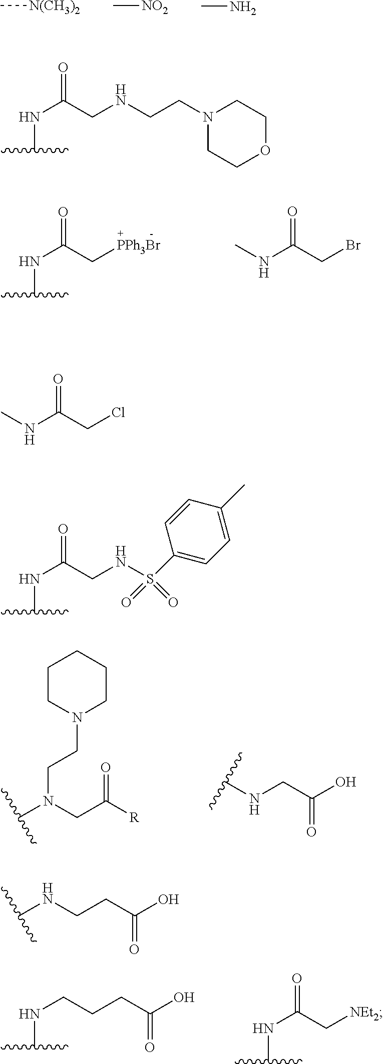

and

R₅=—CO₂Et, —COOH, —CONHNH₂,

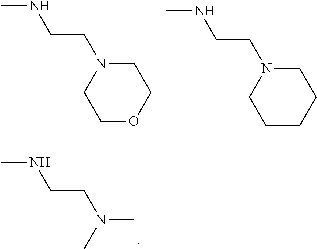

In some illustrative embodiments, this disclosure relates to a diagnostic kit comprising a compound having a formula (I) as disclosed herein, wherein R₂ and R₃ are methyl; R₄ is —N(CH₃)₂, —NO₂, or —NH₂; and R₅ is —CO₂Et.

In some illustrative embodiments, this disclosure relates to a diagnostic kit comprising a compound having a formula (I) as disclosed herein, wherein R₅ is —CO₂Et: R₂ and R₃, independently, are:

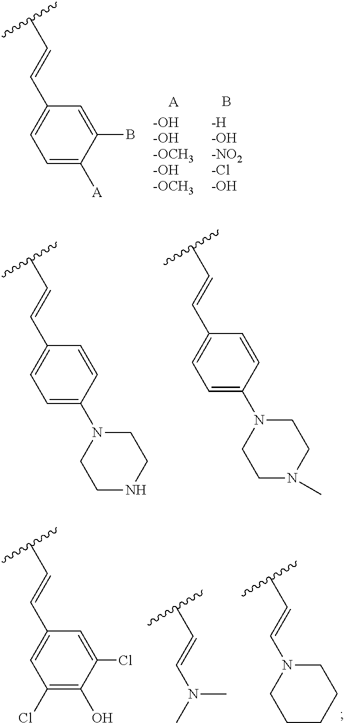

and R₄ is

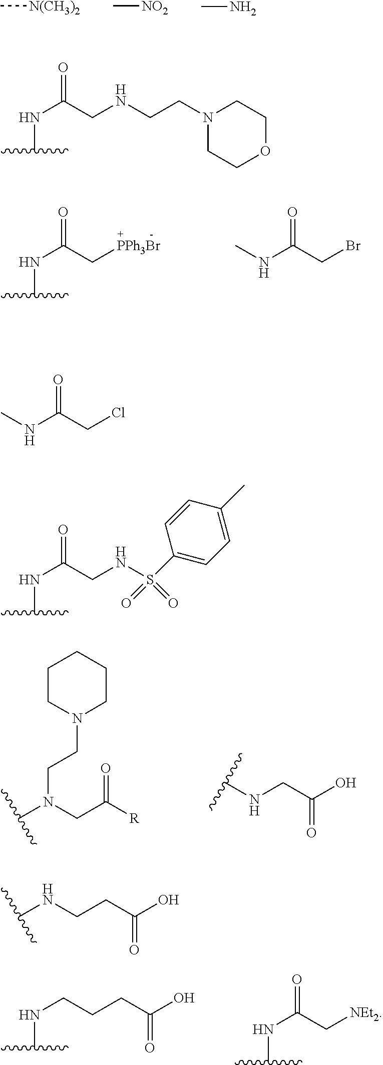

In some illustrative embodiments, this disclosure relates to a diagnostic kit comprising a compound having a formula (I) as disclosed herein, wherein R₂ and R₃ are methyl; R₄ is

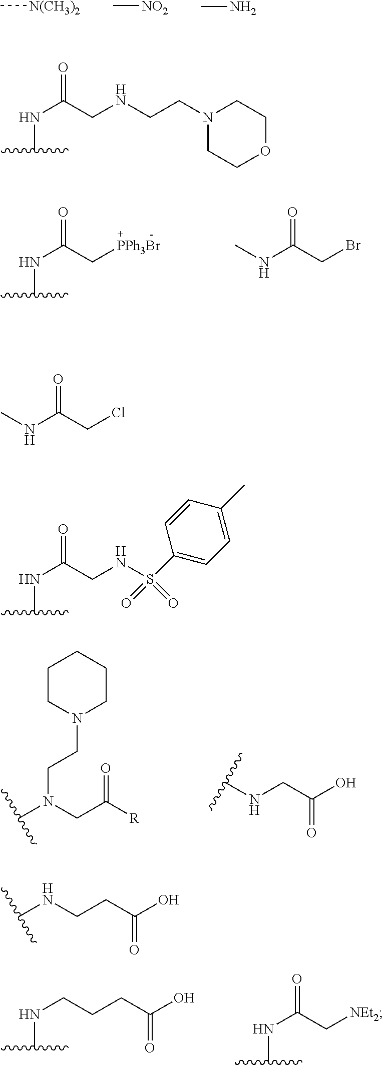

and

R₅=—CO₂Et, —COOH, —CONHNH₂,

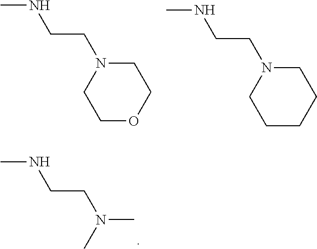

In some illustrative embodiments, this disclosure relates to a diagnostic kit comprising a compound having a formula (I) as disclosed herein, wherein R₅ is —CO₂Et: R₂ and R₃, independently, are:

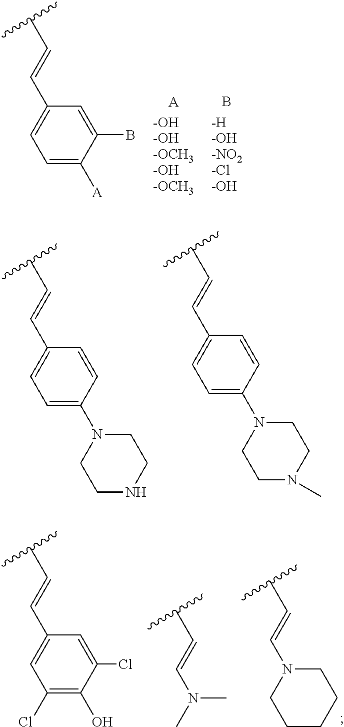

and R₄ is

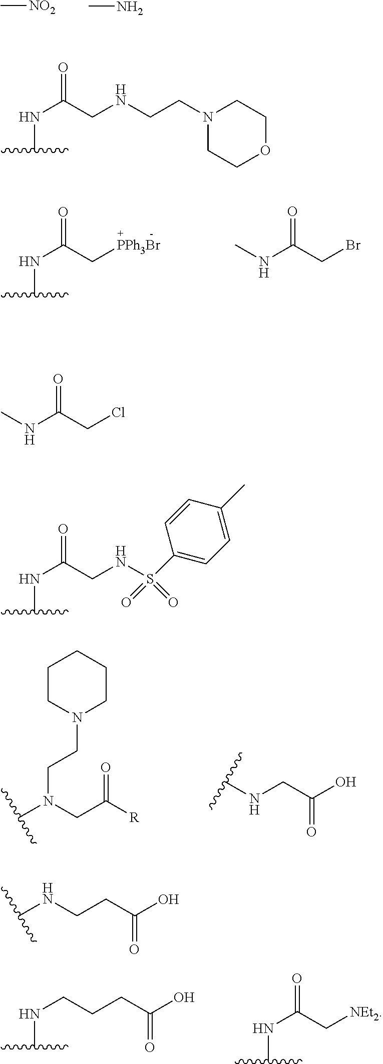

In some illustrative embodiments, this disclosure relates to a diagnostic kit comprising a compound having a formula (I) as disclosed herein, wherein said diagnostic kit is for selective targeting lysosomal, mitochondrial, or nucleus.

In some illustrative embodiments, this disclosure relates to a pH-activable fluorescent probe comprising a compound having a formula (I)

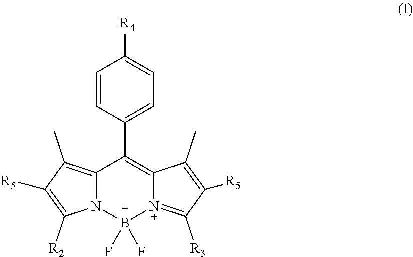

or an acceptable salt thereof, wherein R₂ and R₃, independently, are:

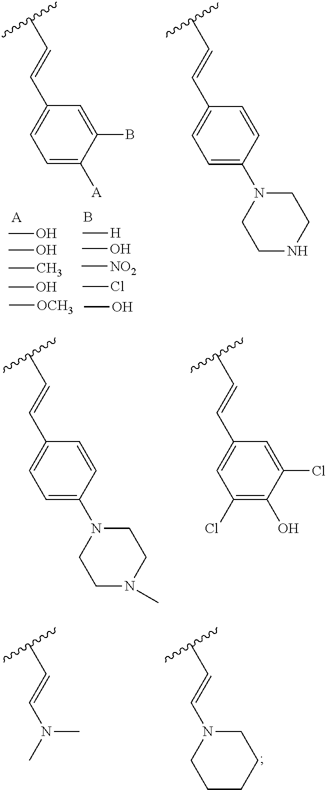

R₄ is

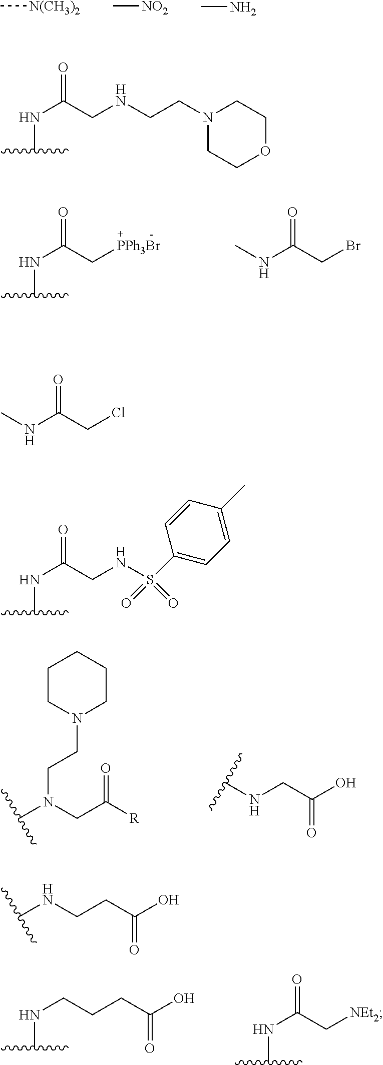

and

R₅=—CO₂Et, —COOH, —CONHNH₂,

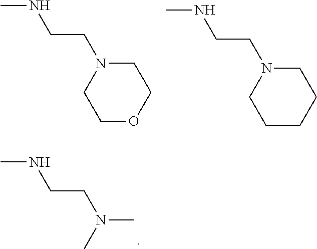

In some illustrative embodiments, this disclosure relates to a pH-activable fluorescent probe comprising a compound having a formula (I) as disclosed herein, wherein R₂ and R₃ are methyl; R₄ is —N(CH₃)₂, —NO₂, or —NH₂; and R₅ is —CO₂Et.

In some illustrative embodiments, this disclosure relates to a pH-activable fluorescent probe comprising a compound having a formula (I) as disclosed herein, wherein R₅ is —CO₂Et; R₂ and R₃, independently, are:

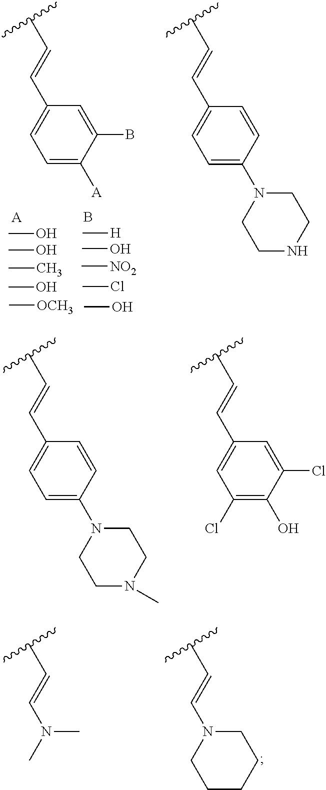

and R₄ is

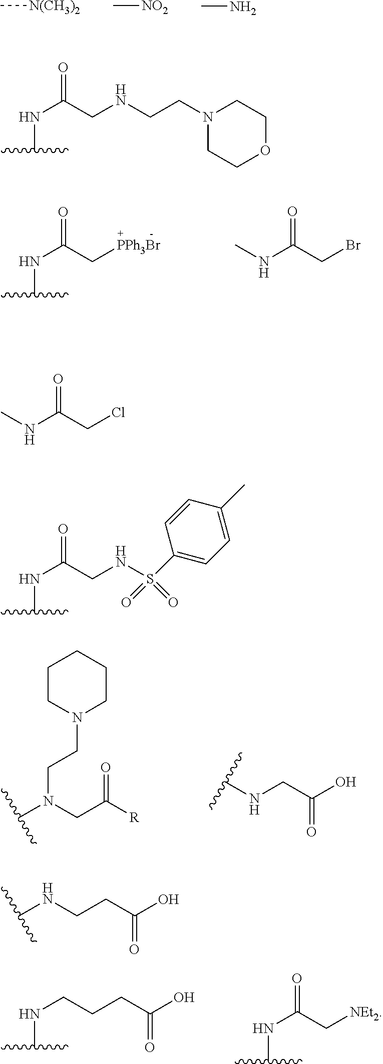

In some illustrative embodiments, this disclosure relates to a pH-activable fluorescent probe comprising a compound having a formula (I) as disclosed herein, wherein R₂ and R₃ are methyl; R₄ is

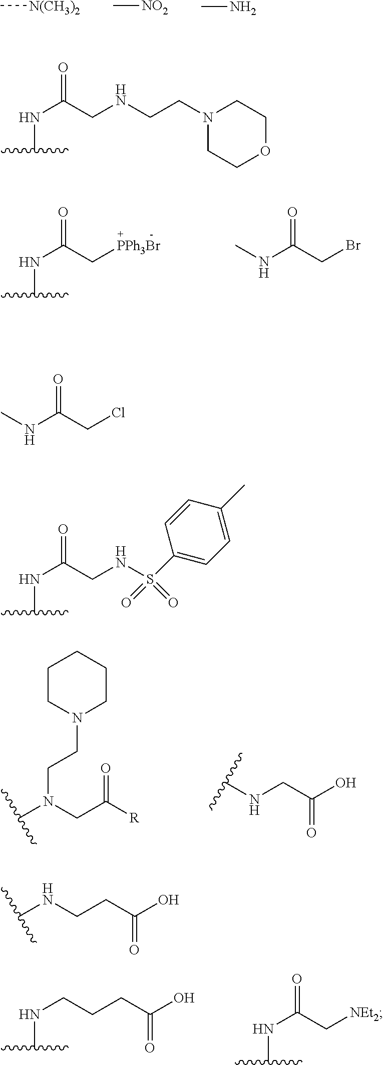

and

R₅=—CO₂Et, —COOH, —CONHNH₂,

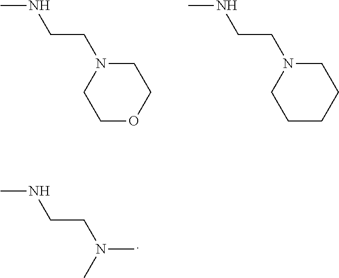

In some illustrative embodiments, this disclosure relates to a pH-activable fluorescent probe comprising a compound having a formula (I) as disclosed herein, wherein R₅ is —CO₂Et; R₂ and R₃, independently, are:

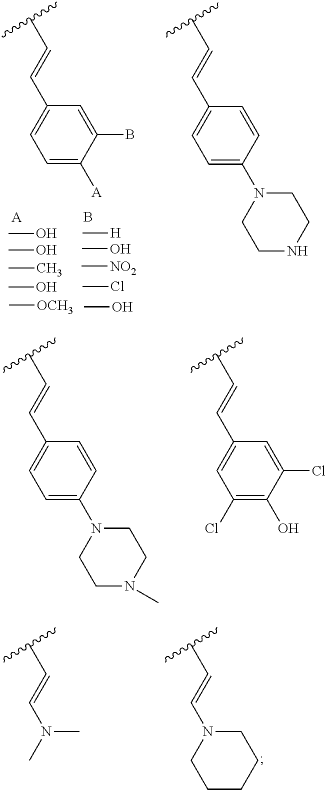

and R₄ is

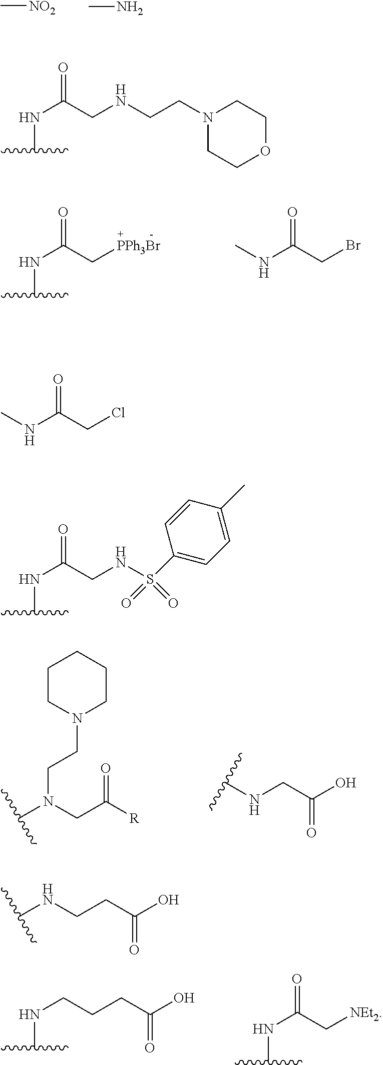

In some illustrative embodiments, this disclosure relates to a pH-activable fluorescent probe comprising a compound having a formula (I) as disclosed herein, wherein said probe is useful for selective targeting lysosomal, mitochondrial, or nucleus.

The following non-limiting exemplary embodiments are included herein to further illustrate the invention. These exemplary embodiments are not intended and should not be interpreted to limit the scope of the invention in any way. It is also to be understood that numerous variations of these exemplary embodiments are contemplated herein.

Live-cell organelle targeted imaging is an important endeavor in understanding ongoing cell processes. In general, fluorescent probes having distinct structures are being used for targeting specific cell organelle. Herein, we aimed to design modular one-step synthetic strategies using a common reaction intermediate to develop new lysosomal, mitochondrial and nucleus targeting pH-activable fluorescent probes based on a single BODIPY scaffold. The divergent cell organelle targeting was achieved by synthesizing pH-activable fluorescent probes with differential fluorescence profiles arising due to the presence of a unique functional group in the scaffold. Further, we also show that how the functional group transformation in the same scaffold influences the localization ability of pH-activable fluorescent probes in cell organelle. The development of pH-activable fluorescent probes that target lysosomes and mitochondria organelles in live and fixed primary mouse microglial cells warrants future use in disease-specific biological models.

The rational design of pH-activable florescent probes should satisfy several parameters: (i) ability to emit high fluorescence at lysosomal acidic pH and negligible fluorescence at cytosolic neutral pH, (ii) cellular permeability and uptake, (iii) non-specific binding to other cellular components, and (iv) good solubility. Several pH-activable fluorescent probes contain rhodamine,^{5a} coumarin,^{5b} napthalimide,^{4b} cyanine^{4e-f} and 4,4-difluoro boron dipyrromethane (known as BODIPY) based scaffolds.^{5c} The widely used are BODIPY-based scaffolds^{5 d} due to a variety of synthetic routes to introduce diverse functionalities^{6a-b} for desired photophysical and spectroscopic properties. However, this process is not robust and minor changes in the substituents can significantly affect spectroscopic properties. Furthermore, if we can develop a synthetic strategy that could furnish divergent cell organelle targeting fluorescent probes, would not only reduce the chemical burden but also afford a convenient synthesis of fluorescent probes from a single synthetic intermediate.

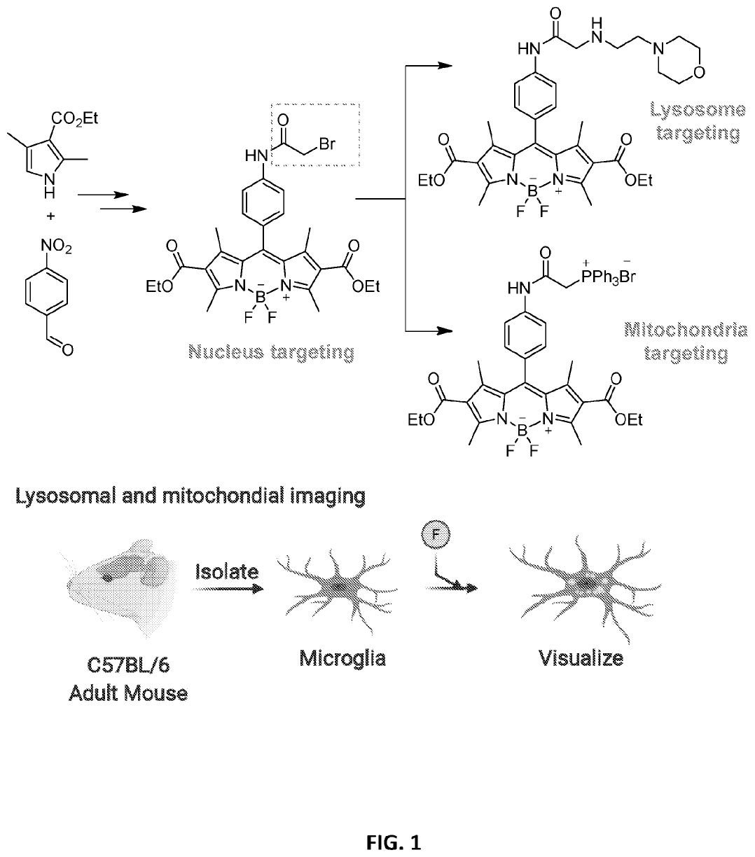

Representative fluorescent probes with different chemical scaffolds that specifically target nucleus (Hoescht 33258),^{2g} lysosome (PhagoGreen)^{6b} or mitochondria (MitoTracker Green™)^{2f} are shown in FIG. 1. Here, we report a new modular design strategy for developing ratiometric BODIPY-based fluorescent probes targeting lysosomes, mitochondria and the nucleus that are highly fluorescent at acidic pH levels compared to cytosolic pH levels (FIG. 1). During the course of the present study, we identified an interesting synthetic intermediate that is an excellent nucleus targeting fluorescent probe. We identified organelle targeting pH-activable fluorescent probes that are cell-permeable, and non-toxic to the cells. One of the most common starting materials to prepare BODIPY probes is 2,4-dimethyl-1H-pyrrole (Scheme 1, compound 1) that exists as a liquid at room temperature. We used ethyl 2,4-dimethyl-1H-pyrrole-3-carboxylate (2) that is solid at room temperature, easy to handle, well-tolerated under reaction conditions, and underrepresented in the literature to prepare boron dipyrromethene scaffold (3).^{6c} The additional functional group on the pyrrole ring system can serve as a handle for late-stage functionalization. Substitutions of BODIPY have significant effects on the excitation/emission property of a fluorescent probe but have only been studied at the 1,3,5,7-positions in scaffold 3, using compound 1 but not compound 2 (Scheme 1).

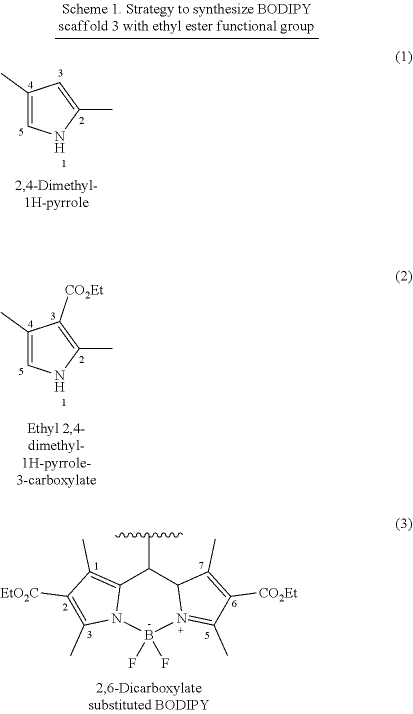

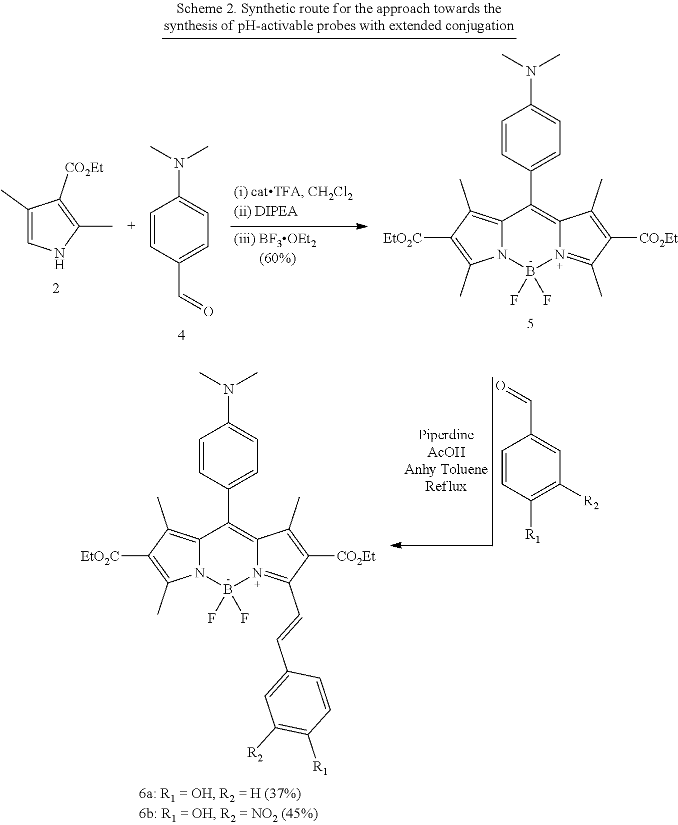

Previous reports suggest that N,N-dimethylaniline functional group can be used to prepare pH-activable probes. Urano and Kobayashi et al.^{7a} and Kikuchi et al.^{7b} reported —NMe₂ containing probe with pKa 4.3 and 4.5, respectively. Therefore, we strategically designed compound 5 to be synthesized in two steps (see Scheme 2) using 2,4-dimethyl-1H-pyrrole-3-carboxylate (2) and 4-(N,N-dimethylamine) benzaldehyde (4). Interestingly, the absorbance and fluorescence property of 5 showed the high fluorescence at pH 2.0 or less that was significantly different from similar probes reported in the literature.^{7a-b} This suggests that the ethylester functional group played a role in the pH-sensitive property of compound 5. Notably, the Knoevenagel condensation between a methyl group at position 3 or 5 of BODIPY scaffold and substituted benzaldehyde was used to introduce extended conjugation to fine-tune the pH-sensitive property towards bathochromic (red) fluorescence shift. Specifically, compound 5 was reacted with 4-hydroxybenzaldehyde or 4-hydroxy 3-nitro benzaldehyde using piperidine, acetic acid as additives, and anhydrous toluene as a solvent under reflux condition to obtain expected conjugated products 6a-b (Scheme 2).^{c,d} However, 6a-b were sparingly soluble in an aqueous medium and 6a did not generate a fluorescence spectrum as solubility was affected by pH buffers. Compound 6b was soluble in a mixture of DMSO: acetonitrile and showed pH-sensitivity with significant fluorescence at pH less than 6 due to the presence of a nitro group at ortho-position to the hydroxyl group.^{7e} However, poor solubility of these compounds does not warrant its use in primary cells and for future in vivo applications.

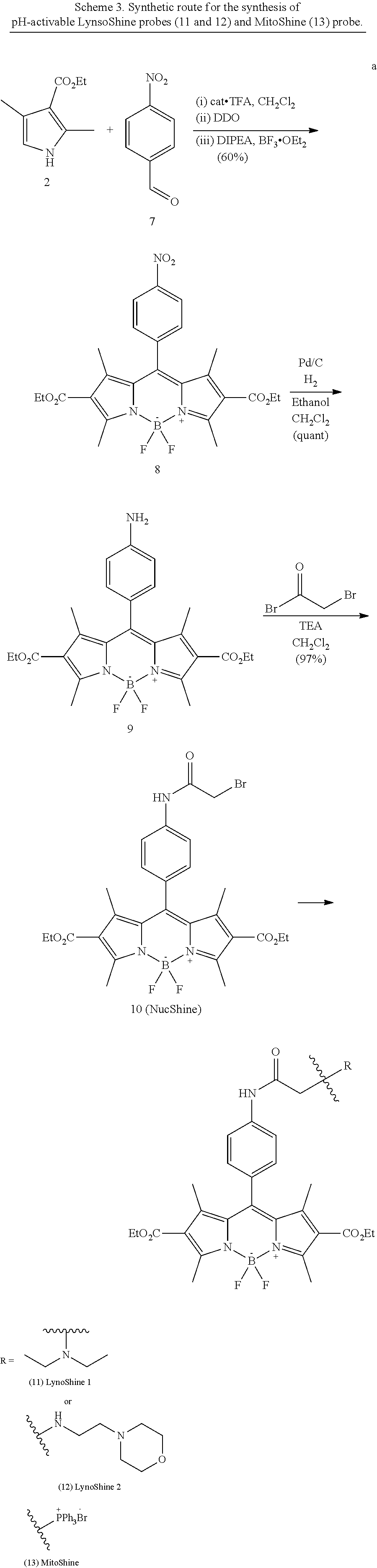

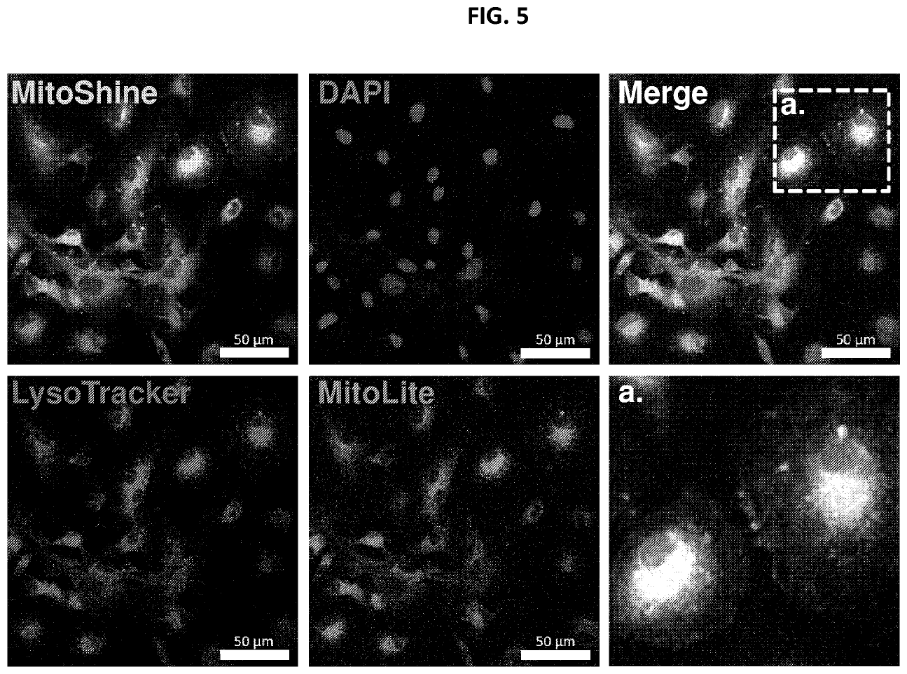

To overcome these challenges, we designed another synthetic route to achieve a facile and modular synthesis of pH-activable fluorescent probes targeting lysosomes, mitochondria and the nucleus (Scheme 3). Herein, we envisioned preparing an important synthetic intermediate that can be transformed into different organelle targeting probes in one step. Using the common synthetic intermediate, we installed functional groups that target a specific cell organelle individually. For example, we planned to synthesize pH-responsive lysosome targeting probes, using morpholine as a lysosome-targeting moiety.^{1a,5c,8a-b} The BODIPY fluorophore can be fine-tuned by the photo-induced electron transfer (PET) mechanism of the lone pair electrons of a nitrogen atom in the morpholine as well as secondary amine functional group (FIG. 6). A similar mechanism can be envisioned when the diethylamine group is present. We started the synthesis of designed pH-responsive probes by the reaction of compound 2 with 4-nitro benzaldehyde (compound 7) afforded compound 8 as a brownish-black solid. The nitro compound 8 was reduced successfully into amine-containing compound 9 using Pd/C in ethanol: CH₂Cl₂ solvent mixture. Next, compound 9 readily reacted with bromo acetyl bromide to give 10 with excellent yields. The CH₂Br handle provides easy access to substitute with an amine functional group-containing reactant to get the final product. We used diethylamine and 2-aminoethyl morpholine substrate to synthesize compounds 11 (LysoShine 1) and 12 (LysoShine 2), respectively. We noticed that CH₂Br in intermediate 10 could serve as a useful synthon to introduce another targeting moiety such as cationic triphenylphosphine for mitochondria targeting. Therefore, we prepared a mitochondrial probe (compound 13) also using intermediate 10 when reacted with triphenylphosphine under the inert condition and named compound 13 as MitoShine.

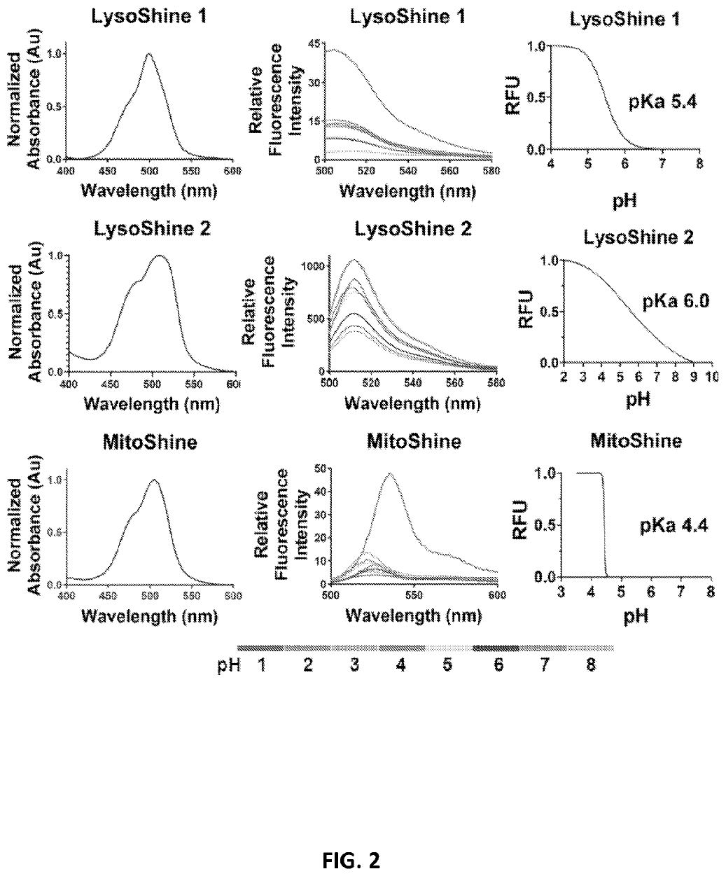

Next, we investigated the absorption and fluorescent properties of LysoShine 1, LysoShine 2, and MitoShine (FIG. 2). The LysoShine 1 in different pH solutions of phosphate buffer (1% DMSO in 1M PBS) has an absorption centered around 500 nm and emission maximum at 505 nm with 480 nm excitation. LysoShine 1 is highly fluorescent at pH 4 compared to reduced fluorescence that gradually decreased from pH 5 to 7 (pKa of 5.4, FIG. 2). On the other hand, LysoShine 2 (max. absorption 500 nm, max. emission 512 nm, excitation 480 nm) showed better pH-sensitivity with a gradual increase for pH less than 6 (pKa of 6.0). We tested the mitochondrial targeting property of MitoShine (max. absorption 505 nm, max. emission 535 nm, excitation 480 nm). Interestingly, MitoShine showed significantly higher fluorescence at pH 4 compared to other pH values (FIG. 2) with a pKa of 4.4 suggesting possible use to image mitochondria-lysosome crosstalk. We also tested the photophysical property of the important synthetic intermediate compound 10 (absorption maximum 505 nm, max, emission 510 nm at excitation 480 nm). Unlike, compound 6a-b, LysoShine 1 and 2, MitoShine maintains its solubility upon addition of pH solutions (final DMSO concentration 1%), so we considered it as compatible for further biological experiments with cells.

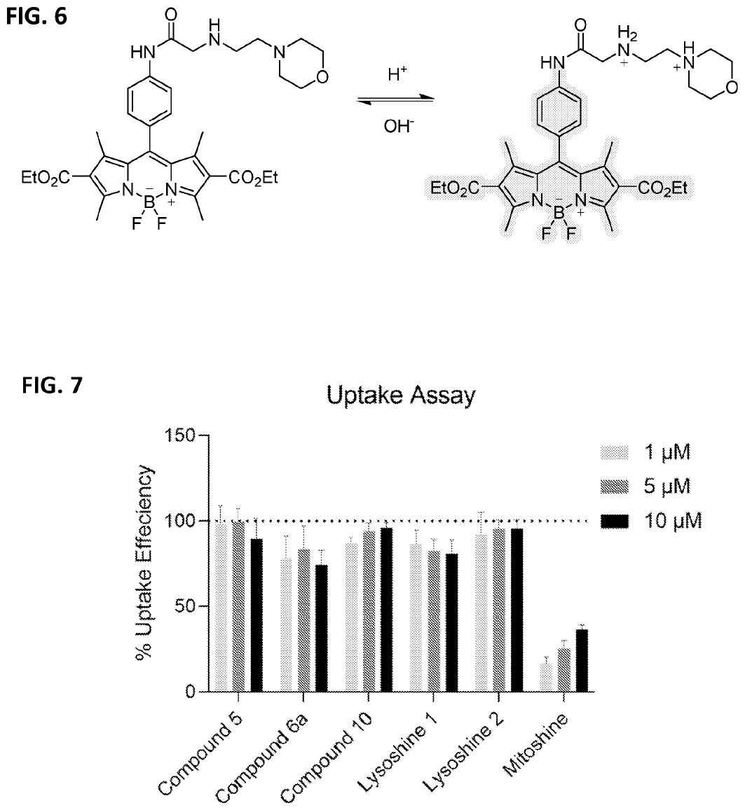

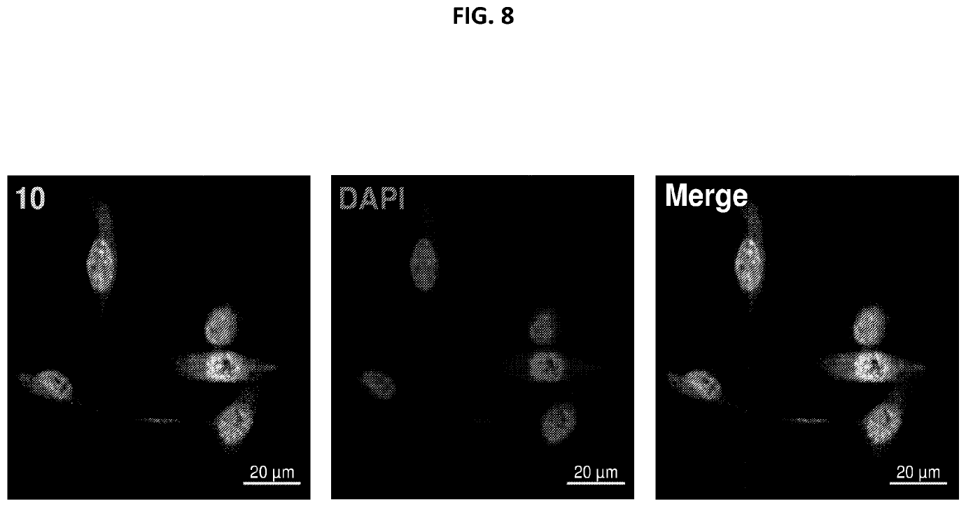

Next, we performed a series of experiments with BV2 microglia cell line and with primary microglia isolated from mouse brains towards applications in biological systems. We performed MTT assay with 1-10 μM concentrations of the probes at 24 hours with BV2 microglia to measure the effect on metabolic activity. All the compounds maintained 80% or higher cellular metabolic activity except for MitoShine, which maintained a high cellular metabolic activity at 1 μM but only 10% activity at 5 μM or higher concentrations. We next assessed the cytotoxicity of the probes on BV2 using the lactate dehydrogenase (LDH) assay that measures the levels of LDH released by dying cells into the cell culture medium. In 2 hour study, LysoShine 1 and 2 did not show any cytotoxicity at any tested doses. Contrarily MitoShine showed no cytotoxicity at 1 and 5 μM, it showed less than 10% cytotoxicity at 10 μM. In 24 hours treatment, LysoShine 1 and 2 showed less than 10% cytotoxicity at all the tested doses compared to MitoShine that showed around 20% cytotoxicity at higher doses of 10 μM and 5 μM. Therefore, the lower metabolic activity of the cells at higher concentrations of MitoShine for a 24 hour period may lead to cytotoxicity. We also checked the uptake efficiency of synthesized probes. In 2 hour treatment, almost all probes showed uptake efficiency around 80% or higher at all doses except for MitoShine that showed 30% of uptake at 10 μM (FIG. 7). Next, having important information (fluorescent property, cytotoxicity and uptake efficiency) in hand, we focused on the cell imaging experiments and flow cytometry analysis to test these pH-activable probes in primary microglia. Notably, compound 10 is one of the important intermediate and found to be cell permeable (FIG. 7) as well as not cytotoxic to BV2 cells. Interestingly, MitoTracker Green has a —CH₂Cl functional group while compound 10 has —CH₂Br. Whereas previously reported microglia specific probes have —CH₂Cl group.^{7f} So, it would be interesting to test the behavior of compound 10 inside the cell. So, first, we checked the localization of compound 10, which showed high fluorescence at pH less than 3. Interestingly, in the confocal imaging, we observed bright green puncta that colocalized with nuclei staining dye DAPI (FIG. 8). For nucleus targeting, DAPI and Hoechst dyes are the most widely used dyes in the field and only a few novel nucleus targeting probes have been reported due to the challenges of nucleus targeting such as poor target efficiency, membrane impermeability etc.^{2f-g} The compound 10 (named NucShine) satisfy these requirements and potentially a new nucleus targeting probe. These observations motivated us to explore—how transforming one functional group to another impacts the cell organelle targeting ability of these pH-activable probes in primary mouse microglia.

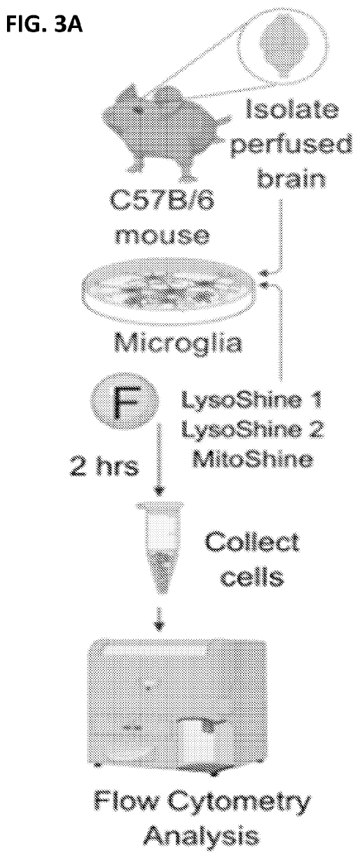

We performed flow cytometry analysis to evaluate the intensity of the fluorescent signals of the probes in primary mouse microglia (FIG. 3a) for future cell sorting applications. The cells treated with the fluorescent probes at 10 μM for 2 hours showed increased green fluorescence compared to the cells treated with the vehicle only (unstained control) thereby clearly discriminating between the probe-treated and untreated cells (FIG. 3b-d). Furthermore, LysoShine 1 showed higher fluorescent intensity than LysoShine 2 within the cells (FIG. 3 along with the commercially available LysoTracker dye, or with (ii) MitoShine probe and the MitoLite dye, we were able to identify over 95% of the live cells that are LysoShine⁺LysoTracker⁺ and around 87% of live cell subset that was MitoShine⁺MitoLite⁺. The ability to identify the probe-specific individual cells also demonstrates the possibility of sorting the cell subsets for downstream analysis in the future.

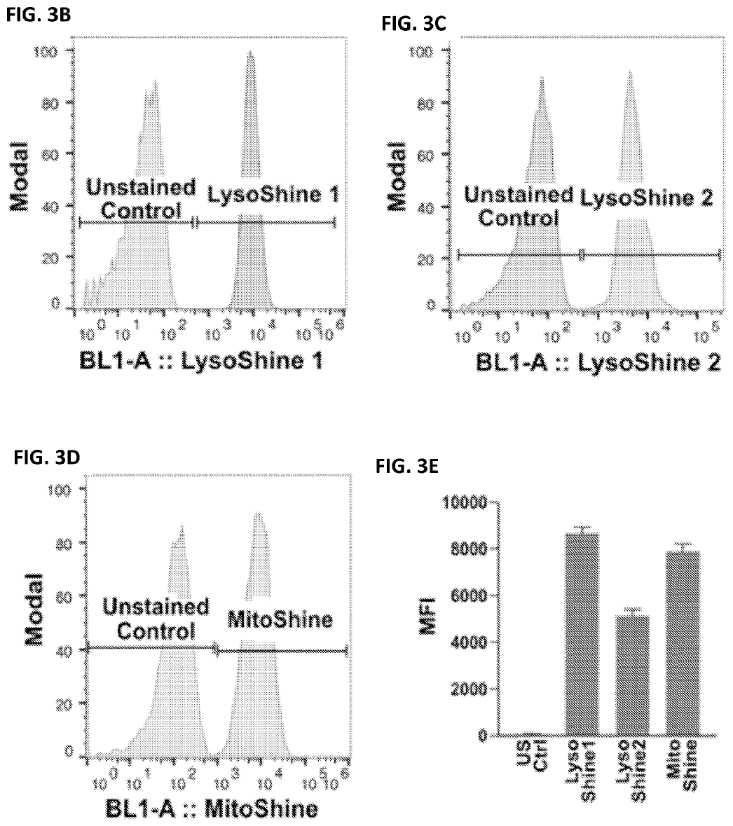

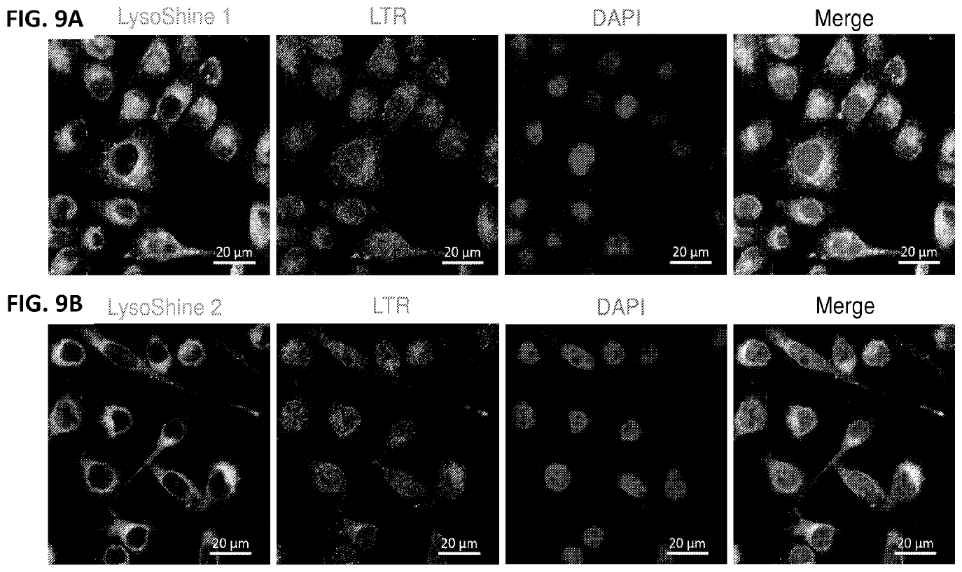

Next, to confirm the localization of the fluorescent probes within the lysosomal organelles, we performed confocal imaging of the probes with primary microglia (FIG. 4a). The green fluorescent signal of LysoShine 1 and LysoShine 2 appeared as several bright puncta around the nuclei as well as in the cytosolic regions of the cells. The localization of the probes into the intracellular acidic organelles was confirmed by co-staining with the commercially available LysoTracker Red DND-99 dye (FIG. 4b-c). We obtained similar fluorescence in BV2 microglia (FIG. 9). The LysoShine probes clearly co-localized with the LysoTracker dye within the acidic lysosomes and not with DAPI-labelled nuclei, confirming targeting in cells.

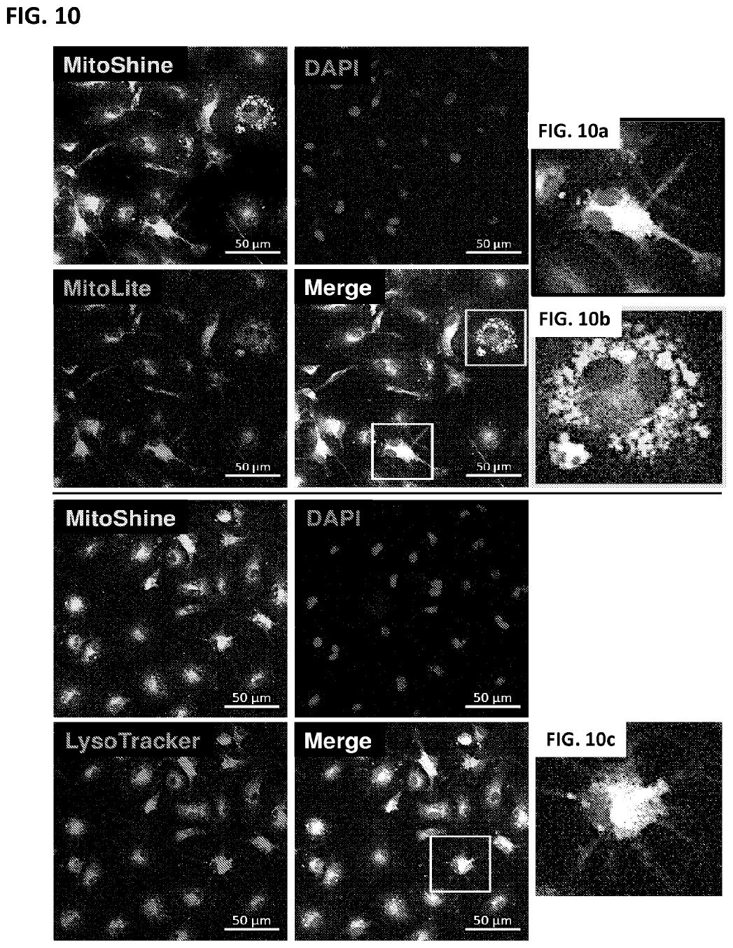

The localization of the mitochondrial probe, MitoShine, was similarly evaluated in primary microglia with confocal imaging. The cells showed a bright green fluorescent signal and the green puncta with MitoShine within the cells clearly co-localized with MitoLite, a commercial mitochondrial dye (FIG. 10a). Interestingly, we observed several other regions (green puncta) labeled with MitoShine that did not overlap with MitoLite (FIG. 10b) suggesting that localization of the MitoShine probe may be localizing in other organelle along with the mitochondria. Interestingly, in a separate experiment, we observed an overlap of the MitoShine probe with LysoTracker, an acidic lysosomal dye (FIG. 10c).

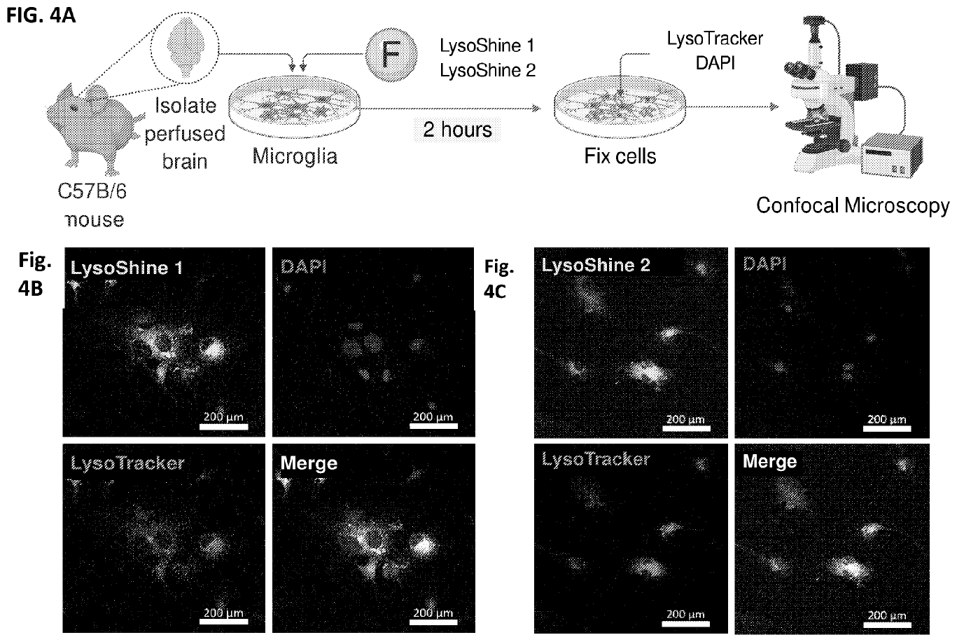

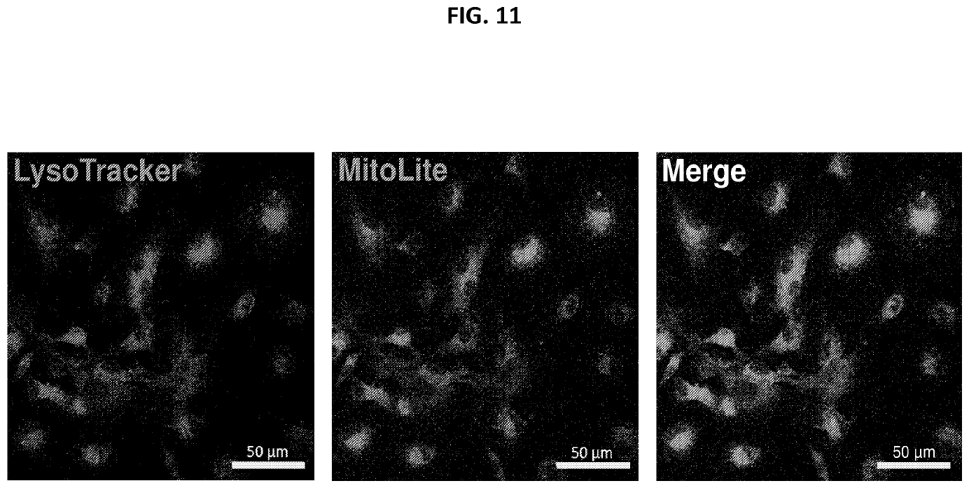

We asked if MitoShine could be used to study mitochondrial transport and recycling as a means to dynamically monitor organelle quality^{4d} or identify mitophagic elimination through lysosomal fusion.^{2e} We therefore treated the primary microglia with MitoShine for 2 hours and later with both LysoTracker and MitoLite at the same time. Confocal imaging demonstrated the colocalization of MitoShine in both mitochondrial as well as in lysosomal organelles (FIG. 5). In addition, overlapping both channels of LysoTracker and MitoLite dyes indicates the merging of mitochondria and lysosomes in these cells (FIG. 11) suggesting the possible use of MitoShine for mitochondrial-lysosomal fusion processes for future biological applications.

In summary, we have developed a modular synthetic strategy for pH-activable fluorescent BODIPY probes to achieve divergent targeting of cellular organelles that are tested in primary cells and available for future biological use in vivo. We showed how the transformation of a critical synthetic intermediate containing bromomethyl group into various derivatives results in a distinct targeting ability of the probe affording lysosomal, mitochondrial and nucleus targeting probes. The synthesized fluorescent probes have high fluorescence at acidic lysosomal pH compared to cytosolic neutral pH. The pH-activable property of fluorescent probes was utilized for targeting lysosomes and mitochondria in primary mouse microglial cells. Besides, the synthetic intermediate or the probes with free amine group can also be used for several bioconjugation reactions, including targeting a protein of interest such as the Aβ(1-42) peptide^{3c,8c} to investigate target-specific microglial uptake towards specific cellular organelles. Further derivatization of pH-activable probes and biological applications will be explored in the future.

### Biological Experimental Section

### Animals

All mice were handled according to the Purdue Animal Care and Use Committee (PACUC) guidelines. Adult C57BL/6 mice (5-7 months old) bred in house were used for isolating microglia.

### Culture and Maintenance of BV2 Microglia

BV2 mouse microglia cells were generously gifted by Dr. Linda J. Van Eldik (University of Kentucky, USA). The BV-2 cell line was developed in the lab of Dr. Elisabetta Blasi at the University of Perugia, Italy. Cells were maintained at 37° C. and 5% CO₂ in DMEM/Hams F-12 50/50 Mix supplemented with 10% Fetal Bovine Serum (FBS), 1% L-Glutamine, and 1% Penicillin/Streptomycin. For imaging experiments, 70,000 cells/2 mL/well were seeded on glass coverslips (Corning #12-553-450) in 6-well plates (Corning #08-772-1B). For flow cytometry experiments, 20,000 cells/0.5 mL were seeded in 24-well plates (Corning #3526).

### Primary Mouse Microglia Isolation and Culture

A detailed protocol for the isolation and culture of primary microglia from adult mouse brains is previously described². Briefly, CD11⁺ primary microglia were isolated from adult C57BL/6 mice aged 5-7 months of age (both male and female sexes) and cultured as follows. Mice were euthanized with CO₂ following the PACUC guidelines, perfused brains were removed and cut into small pieces before homogenizing them in DPBS++with 0.4% DNase-I on the tissue dissociator at 37° C. After filtering the cells through a 70 μm filter, myelin was removed two times, first using gradient centrifugation with Percoll PLUS reagent followed by the use of myelin removal beads on the magnetic column separators. After myelin removal, CD11⁺ cells were selected from the single cell suspension using the CD11⁺ beads as per the manufacturer's instructions. The CD11⁺ cells were finally resuspended in microglia growth media, further diluted in TIC (TGF-β, IL-34, and cholesterol containing) media containing 2% FBS before seeding 0.1×10⁶ cells/500 μL/well of a 24-well plate. The cells were maintained at 37° C. and 10% CO₂ with half-media change every other day until the day of compound treatment (around 12-14 div). For confocal imaging experiments, around 50,000 cells/2 mL cells were sub-cultured at the center of 35 mm glass-bottom imaging dishes (FluoroDish™ #FD35).

### Reconstitution of Fluorescent Probes in DMSO and Cell Treatment

The dried fluorescent probes powders were resuspended in cell grade DMSO to prepare 5 mM stock solutions. This stock was used to make a 1, 5, or 10 μM dilution of the probe in cell culture media. The diluted probe solutions were filtered through 0.22 m filters before adding to the cells.

### Determination of Metabolic Activity of BV2 Microglia With MTT Assay

Murine microglial BV2 cells (10,000 cells/well) were seeded in a 96 well plate and cultured for 24 hours at 37° C. in a 5% CO₂ incubator. Next day, the media was aspirated, and the cells were rinsed twice with PBS (pH 7.4) followed by treatment with 1, 5, or 10 μM of the probe solution made in DMSO (final DMSO concentration=0.05%) for 24 hours in the incubator. Next, the media was aspirated and 20 μL of 5 mg/mL 3-(4,5dimethylthiazol-2-yl)-2,5-diphenyltetrazolium bromide (MTT) solution was added to the cells and then incubated for three hours. The MTT solution was then removed and 100 μL DMSO was added to dissolve the violet formazan crystals. The plate was shaken for 5 minutes using an orbital shaker. The absorbance value at 450 nm was recorded and the percent cell metabolic activity was calculated as the ratio of sample absorbance to control absorbance.

### Determination of Cytotoxicity of the Probes on BV2 Microglia With LDH Assay

The cytotoxicity of the fluorescent probes was determined using the Invitrogen™ CyQUANT™ LDH Cytotoxicity Assay kit which measures the release of Lactate dehydrogenase (LDH) from the dead and dying cells. The assay was performed per the manufacturer's protocol. Briefly, BV2 cells (5000 cells/100 μL/well) were seeded in a 96 well plate and cultured for 24 hours at 37° C. in a 5% CO₂ incubator. After 24 hours, the media was removed, and the cells were treated with 1, 5, or 10 μM of the probes for 2 or 24 hours of incubation. Three additional wells were treated with the given lysis buffer for 45 minutes (positive control). After the corresponding incubation period, the total LDH release was measured using a fluorescent plate reader. The cells without any probe treatment correspond to spontaneous LDH release and were taken as negative control and the cells treated with the lysis buffer correspond to maximum LDH release. The percentage cytotoxicity of the probes was determined as follows:

$$\% cytotoxicity = \frac{\begin{matrix}[ Probe - treated LDH activity - \\ Spontaneous LDH activity ] \times 100\end{matrix}}{\begin{matrix}[ Maximum LDH activity - \\ Spontaneous LDH activity ]\end{matrix}}$$

### Determination of Cellular Uptake of the Fluorescent Probes

Cellular uptake of the florescent probes was determined in-vitro as following: BV2 cells (20,000 cells/well) were seeded in a 96 well plate and cultured overnight at 37° C. in a 5% CO₂ incubator. Next day, the media was removed, and the cells were treated with 1, 5, or 10 μM of the probes for 2 hours. After incubation, the supernatant was collected, and the absorbance was recorded at the respective absorbance maxima of the probe. This was compared with absorbance of the probe in media without cells. The percent cellular uptake was calculated as percentage of the ratio of absorbance of the supernatant to the absorbance of probe solution without cells:

% Uptake Efficiency=1-[Aₛᵤₚₑᵣₙₐₜₐₙₜ×100/A_{probe}]

### Flow Cytometry Analysis

Primary mouse microglia were treated with 1, 5, or 10 μM fluorescent probes for 2 hours for the cells to uptake the probe. The media was then aspirated, and the cells were incubated with 100 nM LysoTracker (1 mM stock from Thermo #L7528) or with 0.5x MitoLite (1000-fold dilution from 500x stock of AAT Bioquest #22678) for 1.5 hours. The media was aspirated and 500 μL/well of cold (4° C.) phosphate buffered saline was added to the cells. The cells were gently detached from the plates and transferred to corresponding vials. Finally, 0.1 ug/mL of DAPI was added to the suspended cells (500 μL volume) for 3 mins before analyzing the cells on the Attune N×T flow cytometer (Invitrogen). All the cells were first gated on the side and forward scatter plot (SSC vs. FSC) followed by gating the live cells using the DAPI nuclear stain. Around 10-20 thousand cells were gated from the live cells in order to analyze the cellular fluorescence signal corresponding to the LysoShine/Lysotracker or Mitoshine/MitoLite probes.

### Confocal Imaging

The localization of the fluorescent probes was visualized using confocal microscopy. The cells were incubated with 10 μM probe for 2 hours and then the media was aspirated. The cells were then incubated with 100 nM LysoTracker (1 mM stock from Thermo #L7528) or with 0.5x MitoLite (1000-fold dilution from 500x stock of AAT Bioquest #22678) for 1.5 hours. The media was aspirated, and the cells were fixed with 4% paraformaldehyde for 10 mins followed by nuclear staining with 1 μM/mL DAPI for 5 mins. For BV2 microglia grown on glass coverslips, the coverslips were removed from the wells and transferred to glass slides with a drop of anti-fade reagent (Thermo Fisher Scientific #P36930). For primary mouse microglia grown in 35 mm glass-bottom dishes, the cells were treated with a few drops of the anti-fade reagent and taken for imaging. The images were captured on a Zeiss LSM 880 Upright Confocal microscope equipped with Plan-Apochromat 20x/0.8 objective.

### EXPERIMENTAL SECTION

Unless noted otherwise, all reagents and solvents were purchased from commercial sources and used as received. All reactions were performed in either round bottom flask with septum or microwave vial with seal septum. The proton (¹H) and carbon (¹³C) NMR spectra were obtained using a 500 MHz using Me₄Si as an internal standard and are reported in δ units. Coupling constants (J values) are reported in Hz. Silica gel column chromatography was performed on Teledyne ISCO (EZprep model) instrument. High-resolution mass spectra (HRMS) were obtained using the electron spray ionization (ESI) technique and as TOF mass analyzer. Organic solvents and starting materials were used as received. The absorption and fluorescence spectra were process by GraphPad Prism software (version 9)

### Synthetic Procedures

### Procedure for the Synthesis of Diethyl 10-(4-(dimethylamino)phenyl)-5,5- difluoro-1,3,7,9-tetramethyl-5H-4λ⁴, 5λ⁴-dipyrrolo[1,2-c:2′,1′-f][1,3,2]diazaborinine-2,8- dicarboxylate (5, kpgc02s254)

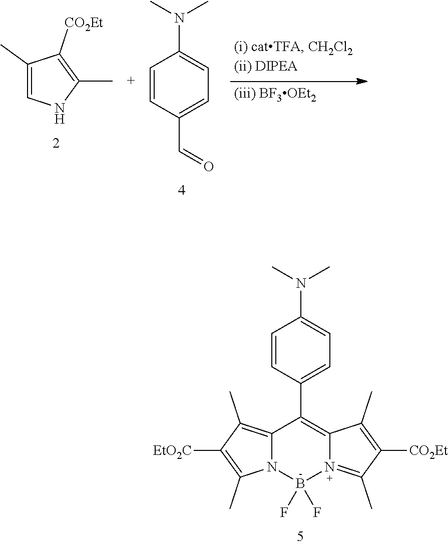

A clean oven dried 500 mL round bottom flask with a magnetic stir bar, charged with 2,4-Dimethyl-1H-pyrrole-3-carboxylic acid ethyl ester (2 equiv., 3 mmol, 502 mg) and 4-(Dimethylamino) benzaldehyde (1 equiv., 1.5 mmol, 224 mg) in anhydrous CH₂Cl₂ (300 mL). The solution was purged with Argon twice. Then, 1 drop of trifluoro acetic acid was added under inert reaction condition and reaction was allowed to stir at room temperature for 24 hours. The reaction was monitored by TLC. Next, septum was removed and DDQ (1 equiv., 1.5 mmol) was added quickly. Again, the reaction mixture was purged with Argon and stirred at room temperature for 15 min. The immediate color change to dark purple was observed. The solvent was partially removed, and compound was isolated over short pad of alumina (neutral) using 1-10% methanol in dichloromethane as an eluent as a dark red solid powder. The product was immediately used for the next step.

In a clean oven dried round bottom flask (500 mL) with a stir bar, a mixture of the resulting compound, anhydrous DCM (200 mL) and diisopropyl ethylamine (5 mL) was added under inert atmosphere. The solution was stirred for 10 minutes. BF₃⋅OEt₂ (5 mL) was added slowly and stirred for additional 5 hours under inert atmosphere. The crude product was extracted with dichloromethane: water, washed with brine, dried over sodium sulfate. Further, compound was purified using flash silica column chromatography with 0-1% methanol in dichloromethane as an eluent and a brownish-purple solid product (460 mg, yield 60%) was obtained. If needed, the purification can be repeated. ¹H NMR (500 MHz, CDCl₃): δ7.03 (d, J=8.8 Hz, 2H), 6.80 (d, J=8.7 Hz, 2H), 4.28 (q, J=7.1, 7.1, 7.1 Hz, 4H), 3.05 (s, 6H), 2.82 (d, J=1.4 Hz, 6H), 1.78 (s, 6H), 1.33 (t, J=7.1, 7.1 Hz, 6H); ¹³C NMR (126 MHz, CDCl₃) δ190.36, 164.52, 158.76, 151.15, 147.76, 147.60, 132.20, 128.80, 122.13, 121.14, 112.43, 111.01, 60.13, 40.23, 40.11, 14.96, 14.32, 14.16; HRMS (ESI) m/z: [M−H]⁺ calcd for C₂₇H₃₁BF₂N₃O₄ 510.2376; Found 510.2380.

### Procedure for the Synthesis of Diethyl (E)-10-(4-(dimethylamino)phenyl)-5,5- difluoro-3-(4-hydroxystyryl)-1,7,9-trimethyl-5H-414,514-dipyrrolo[1,2-c:2′,1′-f][1,3,2]diazaborinine-2,8-dicarboxylate (6a, kpgc02s264)

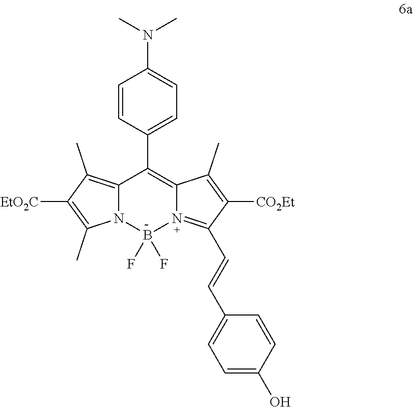 [0110] In a clean oven dried microwave vial with a stir bar, a mixture of compound 5 (30 mg, 0.058 mmol, 1 equiv), 4-hydroxybenzaldehyde (7 mg, 0.058 mmol, 1 equiv), piperidine (100 uL), acetic acid (100 uL), activated molecular sieve (500 mg) were added and the vial was sealed and purged with Argon. Then, anhydrous toluene (2 mL) was added, and reaction mixture was stirred at 120° C. (reflux) for 45 minutes. The reaction was monitored by TLC (Silica, 10% ethylacetate in dichloromethane). The reaction mixture was cooled to room temperature and washed three times with water. The organic phase was dried over sodium sulfate and the organic solvent was evaporated under reduced pressure. The residue was purified by silica gel flash column chromatography (CombiFlash) using 0-20% ethylacetate in dichloromethane to afford the desired compound 6a as reddish-purple solid (13 mg, yield 37%). ¹H NMR (500 MHz, Acetone) δ8.58 (s, 1H), 7.25-7.19 (m, 2H), 6.90 (dd, J=8.7, 4.9 Hz, 4H), 6.71 (d, J=8.6 Hz, 2H), 6.55 (d, J=16.3 Hz, 1H), 5.87 (d, J=16.2 Hz, 1H), 4.25 (dq, J=19.4, 7.1, 7.1, 7.1 Hz, 4H), 3.05 (s, 6H), 2.69 (s, 3H), 1.86 (s, 3H), 1.32 (t, J=7.1, 7.1 Hz, 3H), 1.19 (t, J=7.1, 7.1 Hz, 3H); ¹³C NMR (126 MHz, CDCl₃) δ212.05, 169.69, 168.64, 163.48, 162.65, 161.54, 156.34, 152.20, 151.45, 148.78, 141.03, 136.87, 136.71, 134.73, 133.47, 132.67, 127.02, 125.27, 125.08, 121.64, 120.46, 117.71, 65.61, 65.25, 19.76, 19.25, 19.20, 19.13, 18.83; HRMS (ESI) mlz: [M+H]⁺ calcd for C₃₄H₃₇BF₂N₃O₅ 616.2794; Found 616.2799.

### Procedure for the Synthesis of Diethyl (E)-10-(4-(Dimethylamino)phenyl)-5,5- difluoro-3-(4-hydroxy-3-nitrostyryl)-1,7,9-trimethyl-5H-414,514-dipyrrolo[1,2-c:2′,1′- f][1,3,2]diazaborinine-2,8-dicarboxylate (6b, kpgc02s270)

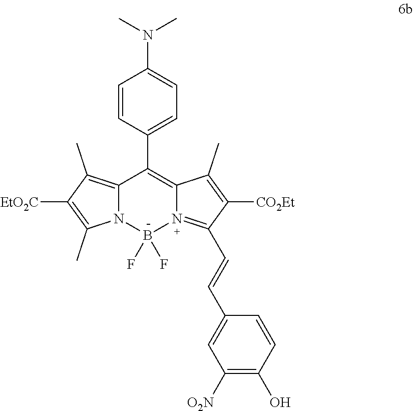

In a clean oven dried microwave vial with a stir bar, a mixture of compound 5 (51.1 mg, 0.1 mmol, 1 equiv), 4-hydroxy-3-nitro benzaldehyde (16.7 mg, 0.1 mmol, 1 equiv), piperidine (100 uL), acetic acid (100 uL), activated molecular sieve (500 mg) were added and purged with Argon. Then, anhydrous toluene (2 mL) was added, and reaction mixture was stirred at 120° C. (reflux) for 12 hours. The reaction was monitored by TLC (Silica, 10% ethylacetate in dichloromethane). The reaction mixture was cooled to room temperature and washed three times with water. The organic phase was dried over sodium sulfate and the organic solvent was evaporated under reduced pressure. The residue was purified by silica gel flash column chromatography (CombiFlash) using 0-30% ethylacetate in dichloromethane to afford the desired compound 6a as dark purple solid (29 mg, yield 45%); ¹H NMR (500 MHz, CDCl₃) δ10.73 (s, 1H), 8.48-8.42 (m, 2H), 8.22 (d, J=2.2 Hz, 1H), 7.92 (dd, J=8.8, 2.2 Hz, 1H), 7.66-7.54 (m, 3H), 7.40 (d, J=16.5 Hz, 1H), 7.22 (d, J=8.8 Hz, 1H), 4.32 (dq, J=18.1, 7.1, 7.1, 7.1 Hz, 4H), 2.88 (s, 3H), 1.67 (s, 3H), 1.54 (s, 6H), 1.32 (dt, J=14.6, 7.1, 7.1 Hz, 6H); ¹³C NMR (126 MHz, CDCl₃) δ164.95, 163.85, 161.44, 155.68, 152.31, 148.79, 147.28, 144.16, 141.15, 137.16, 135.39, 133.66, 131.64, 129.65, 124.84, 124.47, 120.76, 118.20, 61.29, 60.58, 29.72, 15.31, 14.23, 14.11, 13.77; HRMS (ESI) m/z: [M+H]⁺ calcd for C₃₄H₃₆BF₂N₄O₇ 661.2645; Found 661.2652.

### Procedure for the Synthesis of Diethyl 5,5-difluoro-1,3,7,9-tetramethyl-10-(4- nitrophenyl)-5H-4λ⁴,5λ⁴-dipyrrolo[1,2-c:2′, 1′-f][1,3,2]diazaborinine-2,8-dicarboxylate (8, kpgc02s273)

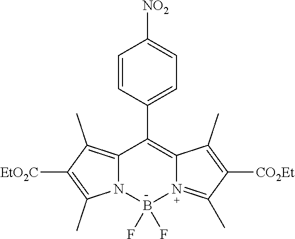 [0114] 4-Nitrobenzaldehyde (2.0 mmol, 302 mg, 1 equiv) and 2,4-Dimethyl-1H-pyrrole-3-carboxylic acid ethyl ester (4.0 mmol, 669 mg, 2 equiv) were dissolved in 350 mL of anhydrous CH₂C1₂ under Argon atmosphere. One drop of TFA was added, and the solution was stirred at room temperature overnight. When TLC monitoring (silica; CH₂C1₂) showed complete consumption of the aldehyde, a solution of 2,3-Dichloro-5,6-dicyano-1,4-benzoquinone (DDQ, 908 mg, 4.0 mmol) in CH₂C1₂ was added, and stirring was continued for 20 minutes under Argon atmosphere. The reaction mixture was washed with water, dried over sodium sulfate, filtered, and evaporated. The compound was purified by short (manual) column chromatography over neutral alumina (CH₂C1₂). The brown powder thus obtained and 5 mL of N,N-Diisopropylethylamine (DIPEA) were dissolved in 200 mL of toluene under an Argon atmosphere. Then 5 mL of BF₃⋅Et₂O was added dropwise, and the solution was stirred at room temperature for 30 min. The reaction mixture was washed with water, dried over sodium sulfate, filtered, and evaporated. The compound was purified by silica gel column chromatography (CH₂C1₂/n-hexane=1/1) to give an orange-red solid (620 mg, yield 60%). ¹H NMR (500 MHz, CDCl₃) δ8.44 (d, J=8.7 Hz, 2H), 7.57-7.52 (m, 2H), 4.29 (q, J=7.2, 7.1, 7.1 Hz, 4H), 2.84 (s, 6H), 1.64 (s, 6H), 1.33 (t, J=7.1, 7.1 Hz, 6H); ¹³C NMR (126 MHz, CDCl₃) δ163.96, 160.60, 148.74, 146.97, 142.28, 141.22, 130.72, 129.52, 124.82, 123.14, 60.48, 15.13, 14.27, 14.02; HRMS (ESI) m/z: [M−H]⁺ calcd for C₂₅H₂₅BF₂N₃O₆ 512.1805; Found 512.1801.

### Procedure¹ for the Synthesis of Diethyl 10-(4-aminophenyl)-5,5-difluoro- 1,3,7,9-tetramethyl-5H-414,514-dipyrrolo[1,2-c:2′, 1′-f][1,3,2]diazaborinine-2,8- dicarboxylate (9, kpgc02s274)

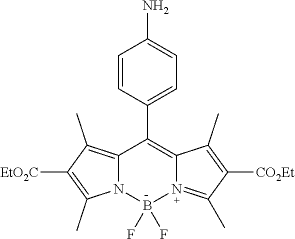 [0116] To a solution of nitro-compound 8 (0.5 mmol) in 120 mL of degassed EtOH:CH₂Cl₂ (1:1), was added a suspension of Pd/C (50 mg, 10% mol) in EtOH under inert atmosphere. The resulting mixture was stirred at room temperature under H₂ (1 atm, balloon) for 12 hours. When the reaction was completed, inorganic solids were removed by filtration through Celite® pad and washed with several portions of CH₂Cl₂. The TLC showed a small amount of impurities. The product was purified by flash chromatography using 0-20% CH₂Cl_{2:} MeOH. The silica in cartridge and column was neutralized by passing acetone (containing 0.5% triethylamine). The orange colored solid was obtained quantitatively. ¹H NMR (500 MHz, CDCl₃) δ6.99 (d, J=8.4 Hz, 2H), 6.81 (d, J=8.4 Hz, 2H), 4.28 (q, J=7.1, 7.1, 7.1 Hz, 4H), 2.82 (s, 6H), 1.78 (s, 6H), 1.33 (t, J=7.1, 7.1 Hz, 6H); ¹³C NMR (126 MHz, CDCl₃) δ164.44, 159.01, 147.82, 146.95, 132.05, 128.91, 123.81, 122.29, 115.69, 60.17, 14.96, 14.31, 14.05; HRMS (ESI) m/z: [M+H]⁺ calcd for C₂₅H₂₉BF₂N₃O₄ 484.2219; Found 484.2225.

### Procedure for the Synthesis of Diethyl 10-(4-(2-bromoacetamido)phenyl)-5,5- difluoro-1,3,7,9-tetramethyl-5H-414,514-dipyrrolo[1,2-c:2′,1′-f][1,3,2]diazaborinine-2,8-dicarboxylate (10, kpgc02s276)

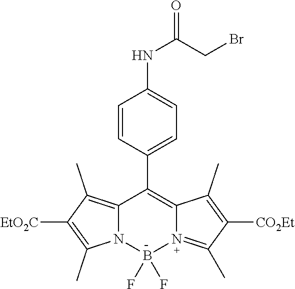 [0118] A solution of NH₂-compound 9 (60 mg, 0.12 mmol) in CH₂Cl₂ (3 mL) was kept in ice-bath. Triethylamine (0.14 mmol, 19 μL) was added to a solution and then bromoacetyl bromide (0.14 mmol, 12 μL) was added slowly. It was stirred at room temperature. Once precipitation was observed within 5 minutes, reaction mixture was directly quenched with 10 mL CH₂Cl₂ and saturated sodium bicarbonate solution. The organic layer was collected and removed under reduced pressure. The product was obtained in quantitative yield as bright orange solid (72 mg, 97% yield). ¹H NMR (500 MHz, CDCl₃): δ8.48 (bs, 1H), 7.82-7.76 (m, 2H), 7.29-7.21 (m, 2H), 4.27 (q, J=7.1, 7.1, 7.1 Hz, 4H), 4.06 (s, 2H), 2.82 (s, 6H), 1.70 (s, 6H). 1.32 (t, J=7.1, 7.1 Hz, 6H); ¹³C NMR (126 MHz, CDCl₃): δ164.28, 163.73, 159.61, 147.57, 145.06, 138.64, 131.56, 130.68, 128.73, 122.59, 120.49, 60.31, 46.23, 29.39, 15.03, 14.28, 13.95; HRMS (ESI) m/z: [M+H]⁺ calcd for C₂₇H₃₀BBrF₂N₃O₅ 604.14030; Found 604.1438.

### Procedure for the Synthesis of Diethyl 10-(4-(2- (diethylamino)acetamido)phenyl)-5,5-difluoro-1,3,7,9-tetramethyl-5H-4λ⁴, 5λ⁴- dipyrrolo[1,2-c:2′, 1′-f][1,3,2]diazaborinine-2,8-dicarboxylate (LysoShine 1, kpgc02s277)

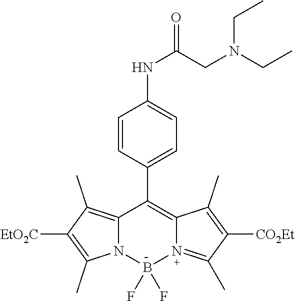 [0120] In a screw cap vial, to a solution of 10 (20 mg, 0.033 mmol) in acetone (1 mL) was added diethylamine (0.066 mmol, 2 equiv, 7 μL) and stirred at 60° C. for 1 hour. The reaction was monitored by TLC (silica, CH₂Cl₂: EtOAc 9:1). Immediately, the reaction mixture was directly loaded on a cartridge and product was eluted in CH₂Cl₂: MeOH (95:5) using flash chromatography. The product was obtained as orange solid (11 mg, 56% yield). ¹H NMR (500 MHz, CDCl₃): δ9.63 (bs, 1H), 7.79 (d, J=8.5 Hz, 2H), 7.22 (d, J=8.5 Hz, 2H), 4.27 (q, J=7.1, 7.1, 7.1 Hz, 4H), 3.19 (s, 2H), 2.82 (s, 6H), 2.69 (q, J=7.1, 7.1, 7.1 Hz, 4H), 2.16 (s, 1H), 1.72 (s, 6H), 1.32 (t, J=7.2, 7.2 Hz, 6H), 1.13 (t, J=7.1, 7.1 Hz, 6H); ¹³C NMR (126 MHz, CDCl₃) 170.50, 164.30, 159.46, 147.65, 145.60, 139.19, 131.67, 129.58, 128.64, 122.51, 119.89, 60.24, 58.13, 48.75, 14.99, 14.29, 14.03, 12.41; HRMS (ESI) m/z: [M+H]⁺ calcd for C₃₁H₄₀BF₂N₄O₅ 597.3060; Found 597.3065.

### Procedure for the Synthesis of Diethyl 5,5-difluoro-1,3,7,9-tetramethyl-10-(4- (2-((2-morpholinoethyl)amino)acetamido)phenyl)-5H-4λ⁴, 5λ⁴-dipyrrolo[1,2-c:2′,1′- f][1,3,2]diaza-borinine-2,8-dicarboxylate (LysoShine 2, kpgc02s280)

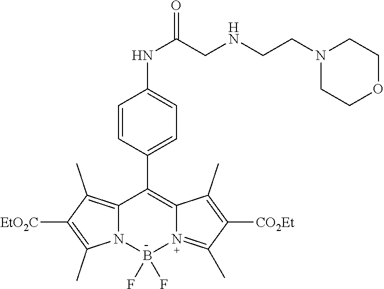 [0122] To a solution of 10 compound (20 mg, 0.033 mmol) in acetone (1 mL) was added 4-(2-aminoethyl)morpholine (0.066 mmol, 2 equiv, ˜9 μL) and stirred at 60° C. for 1 hour. The reaction was monitored by TLC (silica, CH₂Cl₂: EtOAc 9:1) and a new polar product was observed. Immediately, the reaction mixture was directly loaded on a cartridge and product was eluted with gradient of 0-10% methanol in dichloromethane. The product was obtained as Dark orange solid (10 mg, 46% yield). ¹H NMR (500 MHz, CDCl₃) ϵ9.68 (bs, 1H), 7.82 (d, J=8.5 Hz, 2H), 7.22 (d, J=8.5 Hz, 2H), 4.27 (q, J=7.1, 7.1, 7.1 Hz, 4H), 3.71 (t, J=4.7, 4.7 Hz, 4H), 3.43 (s, 2H), 2.82 (s, 8H), 2.56-2.50 (m, 2H), 2.50-2.44 (m, 4H), 1.71 (s, 6H), 1.32 (t, J=7.1, 7.1 Hz, 6H); ¹³C NMR (126 MHz, CDCl₃) δ170.44, 164.30, 159.46, 147.63, 145.55, 139.21, 131.65, 129.63, 128.60, 122.51, 120.06, 66.94, 60.26, 58.11, 53.71, 53.18, 46.39, 15.01, 14.29, 13.95; HRMS (ESI) m/z: [M+H]⁺ calcd for C₃₃H₄₃BF₂N₅O₆ 654.3274; Found 654.3280.

### Procedure for the Synthesis of (2-((4-(2,8-bis(ethoxycarbonyl)-5,5-difluoro- 1,3,7,9-tetramethyl-5H-414,514-dipyrrolo[1,2-c:2′, 1′-f][1,3,2]diazaborinin-10- yl)phenyl)amino)-2-oxoethyl)triphenylphosphonium bromide (MitoShine, kpgc02s286)

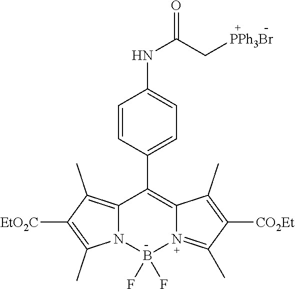 [0124] To a solution of 10 (37 mg, 0.06 mmol) in anhydrous acetonitrile (2 mL), triphenylphoshine (0.18 mmol, 3 equiv,48 mg) was added and stirred at reflux condition overnight. The reaction was monitored by TLC (silica, CH₂Cl₂: EtOAc 9:1) and a new polar product was observed. Immediately, the reaction mixture was directly loaded on a cartridge and product was eluted in CH₂Cl₂: MeOH (90:10). The product was obtained as greenish orange solid (30 mg, 63% yield). ¹H NMR (500 MHz, CDCl₃) δ11.68 (bs, 1H), 7.90-7.76 (m, 10H), 7.70-7.63 (m, 6H), 7.14-7.09 (m, 2H), 5.16 (d, J=14.4 Hz, 2H), 4.28 (q, J=7.1, 7.1, 7.1 Hz, 4H), 2.81 (s, 6H), 2.63 (s, 1H), 2.17 (s, 2H), 1.65 (s, 6H), 1.36-1.30 (m, 6H); ¹³C NMR (126 MHz, CDCl₃) δ164.34, 147.73, 135.27, 134.11, 134.03, 130.34, 130.23, 128.17, 120.83, 117.64, 60.23, 29.28, 14.98, 14.29, 13.85; HRMS (ESI) m/z: [M−Br]⁺ calcd for C₄₅H₄₄BF₂N₃O₅P⁺ 786.3074; Found 786.3079.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### CITED REFERENCES

1. (a) J. Zhang, R. E. Campbell, A. Y. Ting and R. Y. Tsien, Nat. Rev. Mol. Cell Biol., 2002, 3, 906-918; (b) T. Kowada, H. Maeda and K. Kikuchi, Chem. Soc. Rev., 2015, 44, 4953-4972.
2. (a) J. Han and K. Burgess, Chem. Rev., 2010, 110, 2709-2728; (b) J. T. Hou, W. X. Ren, K. Li, J. Seo, A. Sharma, X. Q. Yu, J. S. Kim, Chem. Soc. Rev., 2017, 46, 2076-90; (c) A. R. Sarkar, C. H. Heo, L. Xu, H. W. Lee, H. Y. Si, J. W. Byun, H. M. Kim, Chem. Sci., 2016, 7, 766-73; (2d) L. Fan, X. Wang, J. Ge, F. Li, C. Zhang, B. Lin, S. Shuang and C. Dong, Chem. Commun., 2019, 55, 6685-88; (2e) C. M. Deus, K. F. Yambire, P. J. Oliveira, N. Raimundo, Trends Mol. Med., 2020, 26, 71-88; (2f) Gao P., Pan W., Li N. and Tang B. Chem. Sci., 2019, 10, 6035-6071; (2g) Bucevičius, J., Lukinavičius, G. and Gerasimaitė, R., Chemosensors 2018, 6, 18.
3. (a) S. Gordon, Immunity, 2016, 44, 463-475; (3b) H. Xu and D. Ren, Annu. Rev. Physiol., 2015, 77, 57-80; (3c) P. Prakash, K. P. Jethava, N. Korte, P. Izquierdo, E. Favuzzi, I. Rose, K. A. Guttenplan, S. Dutta, C. Rochet, G. Fishell, S. Liddelow, D. Attwell and G. Chopra, bioRxiv, DOI:10.1101/2020.03.29.002857; (3d) J. B. Zuchero and B. A. Barres, Development, 2015, 142, 3805-3809; (3e) D. M. Paresce, H. Chung and F. R. Maxfield, J. Biol. Chem., 1997, 272, 29390-7; (3f) A. U. Joshi, P. S. Minhas, S. A. Liddelow, B. Haileselassie, K. I. Andreasson, G. W. Dorn and D. Mochly-Rosen, Nat. Neurosci., 2019, 22, 1635-1648.
4. (a) R. E. Lawrence and R. Zoncu, Nat. Cell Biol., 2019, 21, 133-142; (b) X. Liu, L. Wang, T. Bing, N. Zhang and D. Shangguan, ACS Appl. Bio Mater., 2019, 2, 1368-1375; (c) M. H. Lee, N. Park, C. Yi, J. H. Han, J. H. Hong, K. P. Kim, D. H. Kang, J. L. Sessler, C. Kang and J. S. Kim, J. Am. Chem. Soc., 2014, 136, 14136-14142; (d) M. I. Sánchez, Y. Vida, E. Pérez-Inestrosa, J. L. Mascareñas, M. E. Vázquez, A. Sugiura and J. Martínez-Costas, Sci. Rep., 2020, 10, 3528; (e) Mu H., Miki K., Harada H., Tanaka K., Nogita K., and Ohe K., ACS Sens. 2021, 6, 1, 123-129; (f) Cooper, M.E., Gregory, S., Adie, E., and Kalinka S., J. Fluoresc., 2002, 12, 425-429.
5. (a) Y. Zhang, S. Xia, M. Fang, W. Mazi, Y. Zeng, T. Johnston, A. Pap, R. L. Luck and H. Liu, Chem. Commun., 2018, 54, 7625-7628; (b) D. Cao, Z. Liu, P. Verwilst, S. Koo, P. Jangjili, J. S. Kim and W. Lin, Chem. Rev., 2019, 119, 10403-10519; (c) J. L. Zhu, Z. Xu, Y. Yang and L. Xu, Chem. Commun., 2019, 6629-6671; (d) H. Lu, J. MacK, Y. Yang and Z. Shen, Chem. Soc. Rev., 2014, 43, 4778-4823.
6. (a) N. Boens, V. Leen and W. Dehaen, Chem. Soc. Rev., 2012, 41, 1130-1172; (b) A. Vázquez-Romero, N. Kielland, M. J. Arévalo, S. Preciado, R. J. Mellanby, Y. Feng, R. Lavilla and M. Vendrell, J. Am. Chem. Soc., 2013, 135, 16018-16021; (c) N. O. Didukh, V. P. Yakubovskyi, Y. V. Zatsikha, G. T. Rohde, V. N. Nemykin and Y. P. Kovtun, J. Org. Chem., 2019, 84, 2133-2147.
7. (a) Y. Urano, D. Asanuma, Y. Hama, Y. Koyama, T. Barrett, M. Kamiya, T. Nagano, T. Watanabe, A. Hasegawa, P. L. Choyke and H. Kobayashi, Nat. Med., 2009, 15, 104-109; (b) T. Kowada, J. Kikuta, A. Kubo, M. Ishii, H. Maeda, S. Mizukami and K. Kikuchi, J. Am. Chem. Soc, 2011, 133, 17772-17776; (c) H. Xiong, P. Kos, Y. Yan, K. Zhou, J. B. Miller, S. Elkassih and D. J. Siegwart, Bioconjugate Chem., 2016, 27, 1737-1744; (d) M. H. Chua, T. Kim, Z. L. Lim, T. Y. Gopalakrishna, Y. Ni, J. Xu, D. Kim and J. Wu, Chem.-A Eur. J., 2018, 24, 2232-2241; (e) M. Grossi, M. Morgunova, S. Cheung, D. Scholz, E. Conroy, M. Terrile, A. Panarella, J. C. Simpson, W. M. Gallagher and D. F. O'Shea, Nat. Commun., 2016, 7, 1-13; (f) Leong C., Lee S. C., Ock J., Li X., See P., Park S. J., Ginhoux F., Yun S. W., Chang Y. T., Chem. Commun. 2014, 50(9), 1089-1091.
8. (a) S. L. Shen, X. P. Chen, X. F. Zhang, J. Y. Miao and B. X. Zhao, J. Mater. Chem. B, 2015, 3, 919-925; (b) T. H. Chen, S. Zhang, M. Jaishi, R. Adhikari, J. Bi, M. Fang, S. Xia, Y. Zhang, R. L. Luck, R. Pati, H. M. Lee, F. T. Luo, A. Tiwari and H. Liu, ACS Appl. Bio Mater., 2018, 1, 549-560; (c) P. Prakash, T. C. Lantz, K. P. Jethava, G. Chopra, Methods Protoc. 2019, 2 (2), 48.
9. Prakash, P.; Jethava, K. P.; Korte, N.; Izquierdo, P.; Favuzzi, E.; Rose, I.; Guttenplan, K. A.; Dutta, S.; Rochet, C.; Fishell, G.; et al. Monitoring Phagocytic Uptake of Amyloid β into Glial Cell Lysosomes in Real Time. bioRxiv 2020.
10. Zhang, J.; Yang, M.; Li, C.; Dorh, N.; Xie, F.; Luo, F.-T.; Tiwari, A.; Liu, H. Near-Infrared Fluorescent Probes Based on Piperazine-Functionalized BODIPY Dyes for Sensitive Detection of Lysosomal PH. J. Mater. Chem. B 2015, 3 (10), 2173-2184.

What is claimed is:

1. A compound having a formula (I) 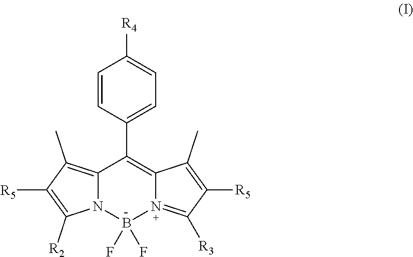
or an acceptable salt thereof, wherein R₂ and R₃, independently, are selected from the group consisting of: 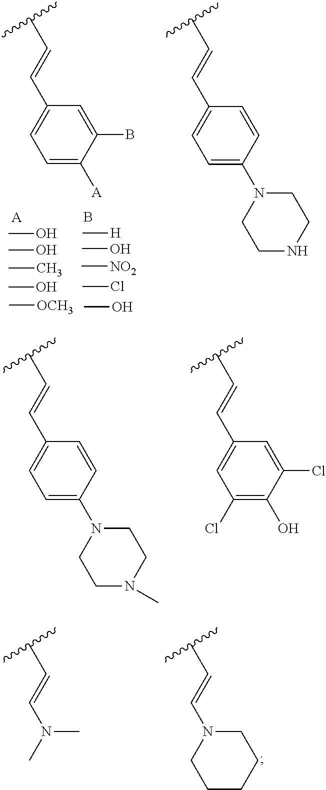
R₄ is selected from the group consisting of: 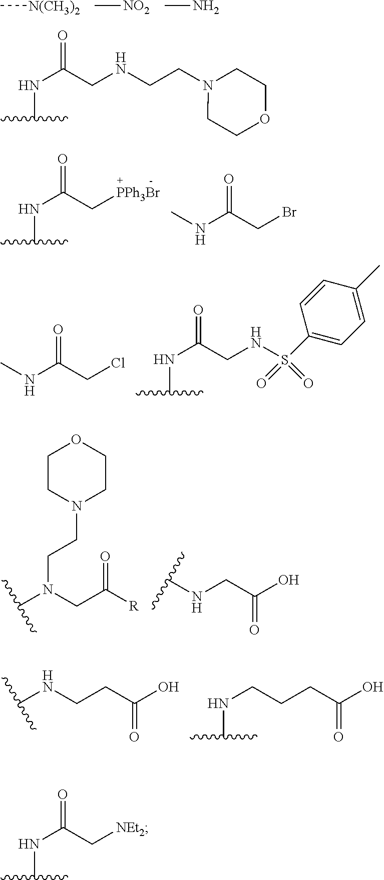
and
R₅ is selected from the group consisting of:
—CO₂Et, —COOH, —CONHNH₂,

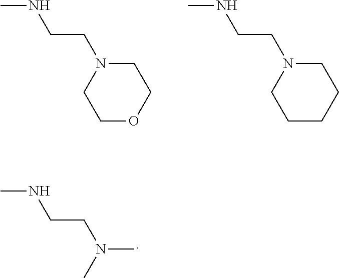

2. The compound according to claim 1, wherein R₂ and R₃ are methyl; R₄ is —N(CH₃)₂,
—NO₂, or —NH₂; and R₅ is —CO₂Et.

3. The compound according to claim 1, wherein R₅ is —CO₂Et; R₂ and R₃, independently, are selected from the group consisting of: 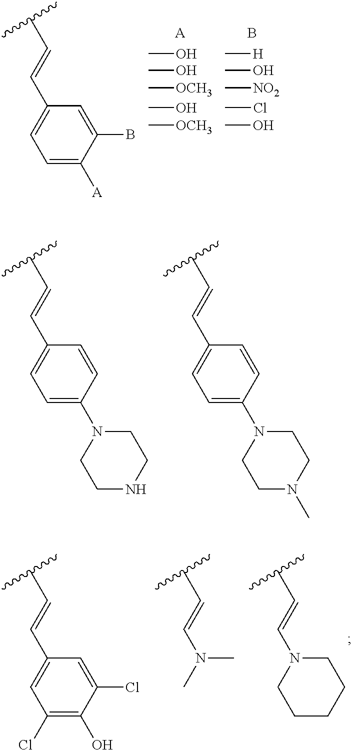
and R₄ is selected from the group consisting of:

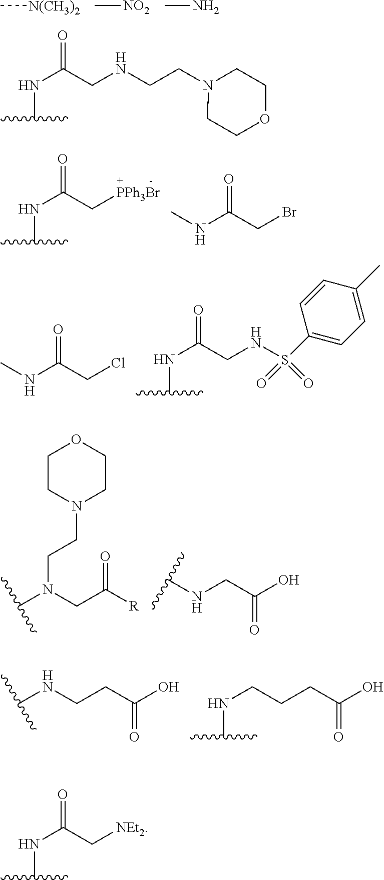

4. The compound according to claim 1, wherein R₂ and R₃ are methyl; R₄ is selected from the group consisting of: 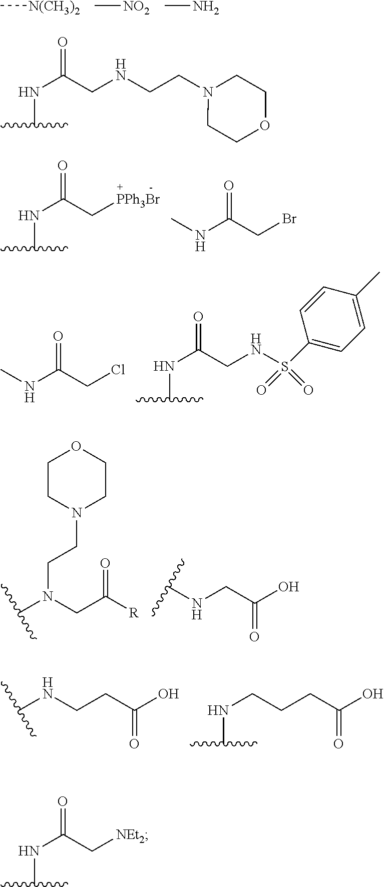
and R₅ is selected from the group consisting of:
—CO₂Et, —COOH, —CONHNH₂,

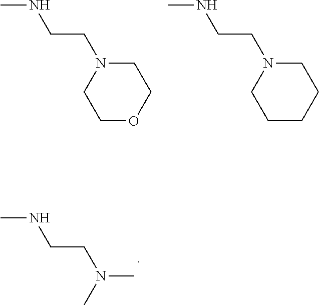

5. The compound according to claim 1, wherein R₅ is —CO₂Et; R₂ and R₃, independently, are selected from the group consisting of: 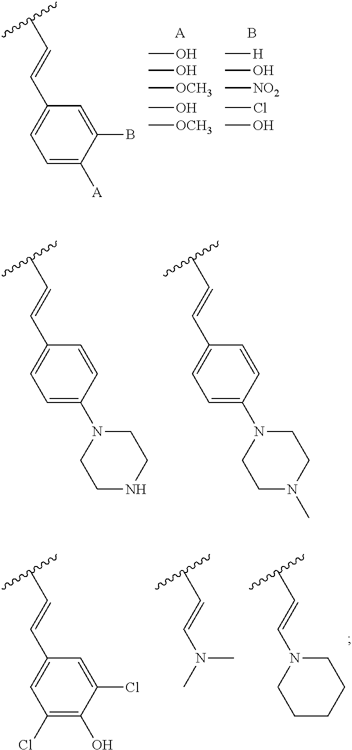
and R₄ is selected from the group consisting of:

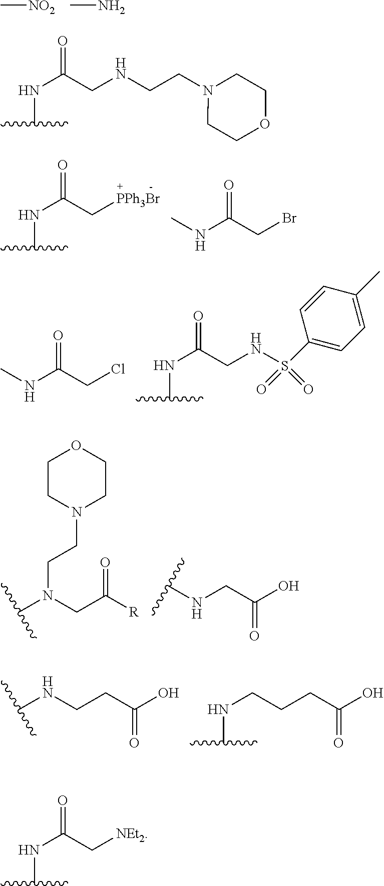

6. The compound according to claims 1, wherein said compounds are useful for as a lysosomal, mitochondrial, and nucleus targeting pH-activable fluorescent probe.

7. A diagnostic kit comprising one or more compounds of claims 1.

8. A diagnostic kit for imaging comprising one or more compounds of claims 1.

9. A diagnostic kit for targeting lysosomal, mitochondrial, or nucleus comprising one or more compounds of claims 1.

10. A pH-probe for diagnostic purpose comprising one or more compounds of claims 1.

11. A pH-probe for imaging comprising one or more compounds of claims 1.

12. A pH-activable fluorescent probe comprising one or more compound of claims 1.

13. A pH-activable fluorescent probe targeting lysosomal, mitochondrial, or nucleus comprising one or more compounds of claims 1.

14. A diagnostic kit comprising a compound having a formula (I) 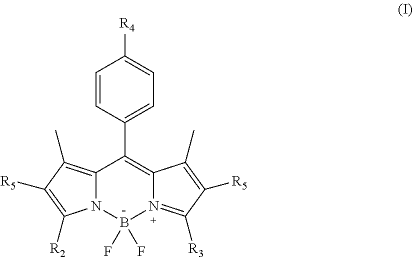
or an acceptable salt thereof, wherein R₂ and R₃, independently, are selected from the group consisting of: 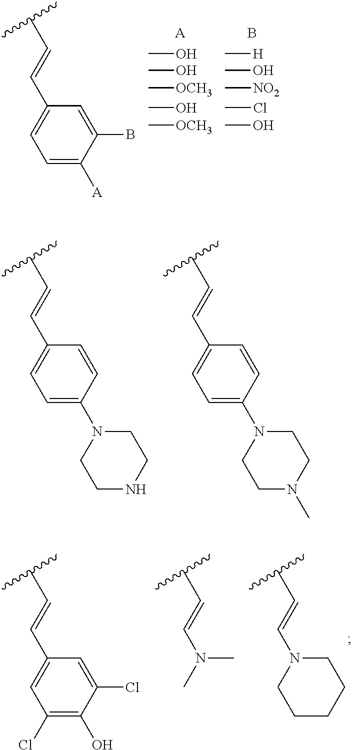
R₄ is selected from the group consisting of: 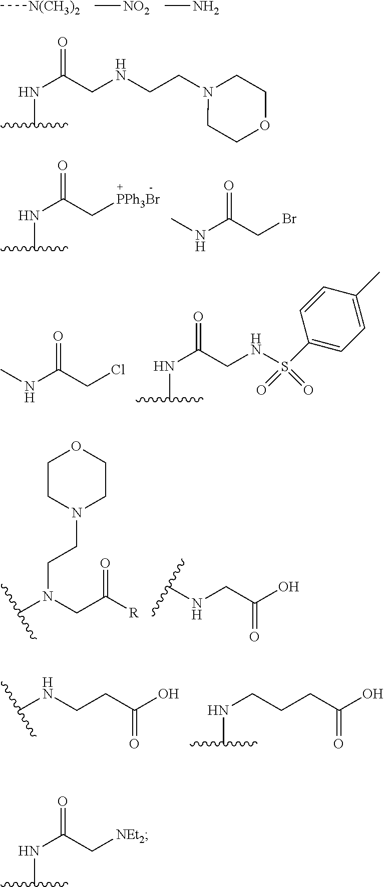
and R₅ is selected from the group consisting of:
—CO₂Et, —COOH, —CONHNH₂,

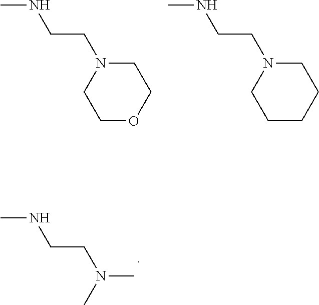

15. The diagnostic kit according to claim 14, wherein R₂ and R₃ are methyl; R₄ is —N(CH₃)₂, —NO₂, or —NH₂; and R₅ is —CO₂Et.

16. The diagnostic kit according to claim 14, wherein R₅ is —CO₂Et; R₂ and R3, independently, are selected from the group consisting of: 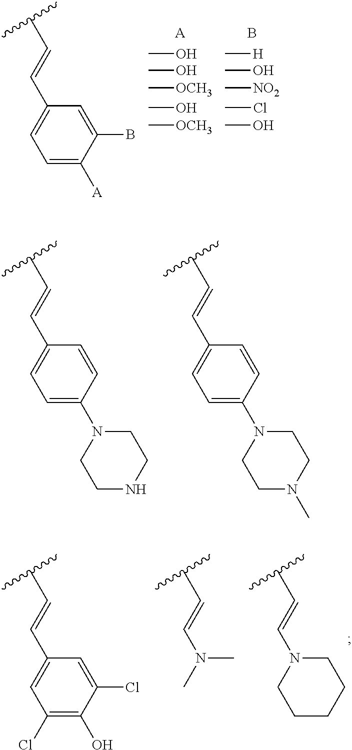
and R₄ is selected from the group consisting of:

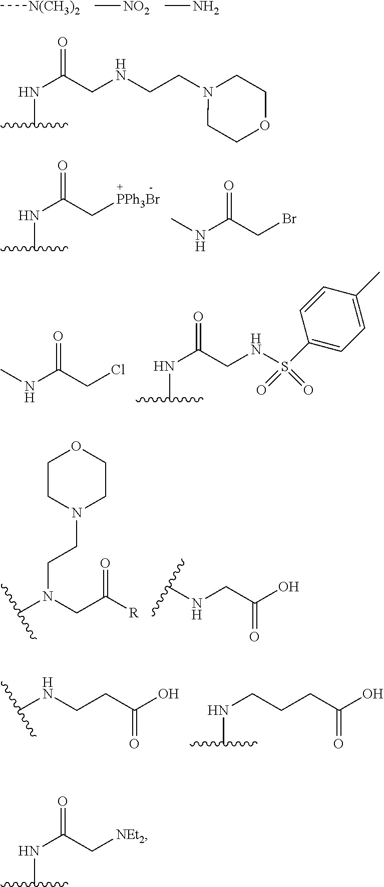

17. The diagnostic kit according to claim 14, wherein R₂ and R₃ are methyl; R₄ is selected from the group consisting of: 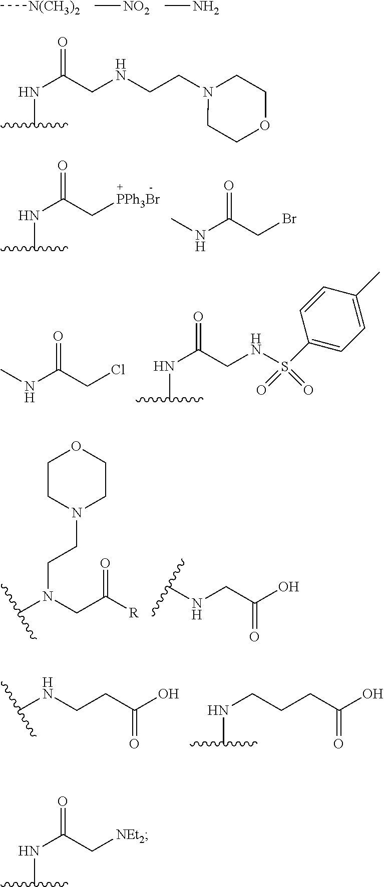
and R₅ is selected from the group consisting of:
—CO₂Et, —COOH, —CONHNH₂,

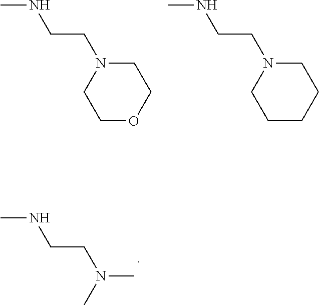

18. The diagnostic kit according to claim 14, wherein R₅ is —CO₂Et; R₂ and R₃, independently, are selected from the group consisting of: 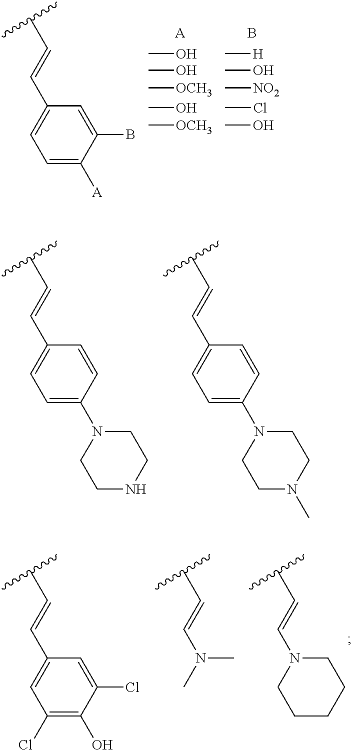
and R₄ is selected from the group consisting of:

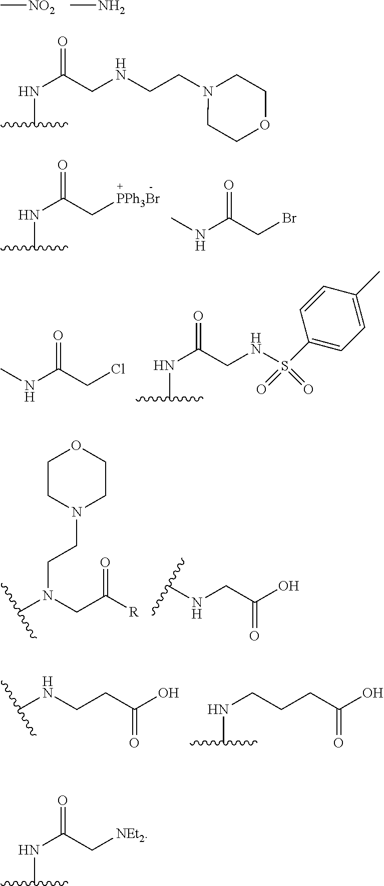

19. The diagnostic kit according to claim 14, wherein said diagnostic kit is for selective targeting lysosomal, mitochondrial, or nucleus.

20. A pH-activable fluorescent probe comprising a compound having a formula (I) 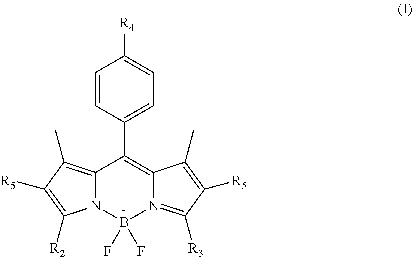
or an acceptable salt thereof, wherein R₂ and R₃, independently, are selected from the group consisting of: 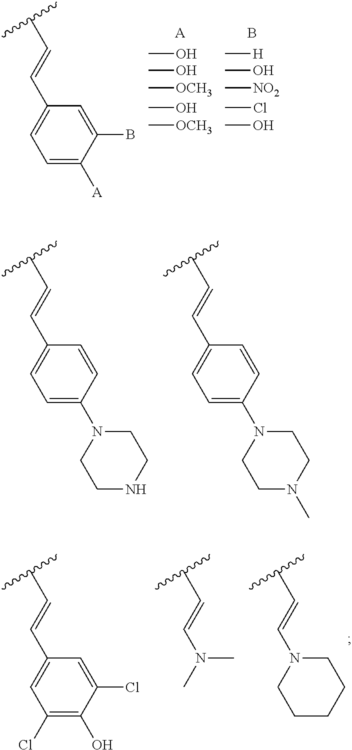
R₄ is selected from the group consisting of: 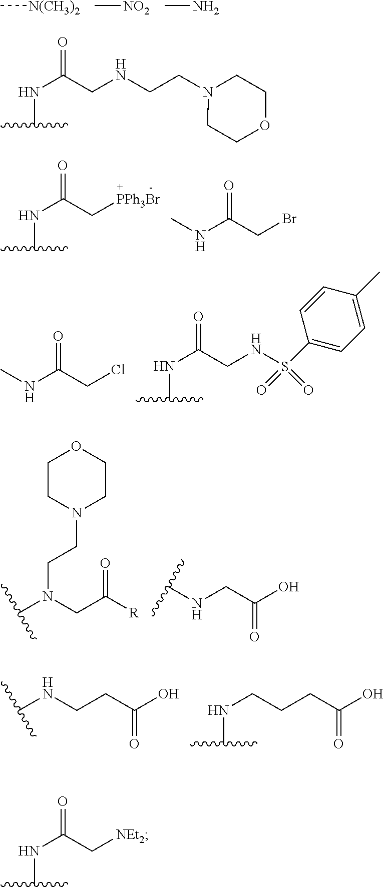
and R₅ is selected from the group consisting of:
—CO₂Et, —COOH, —CONHNH₂,

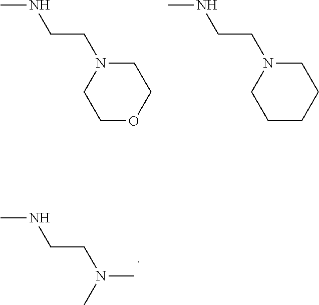

21. The pH-activable fluorescent probe according to claim 20, wherein R₂ and R₃ are methyl; R₄ is —N(CH₃)₂, —NO₂, or —NH₂; and R₅ is —CO₂Et.

22. The pH-activable fluorescent probe according to claim 20, wherein R₅ is —CO₂Et; R₂ and R₃, independently, are selected from the group consisting of: 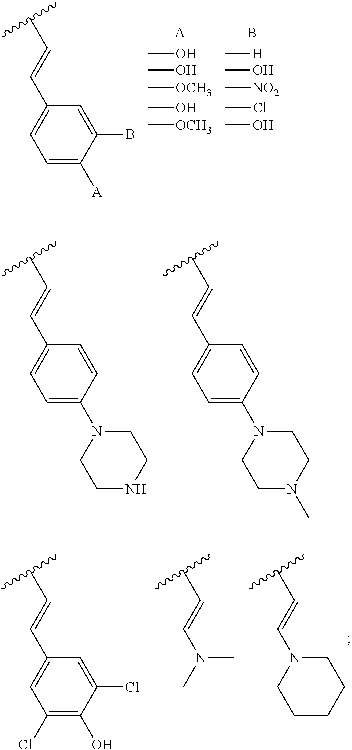
and R₄ is selected from the group consisting of:

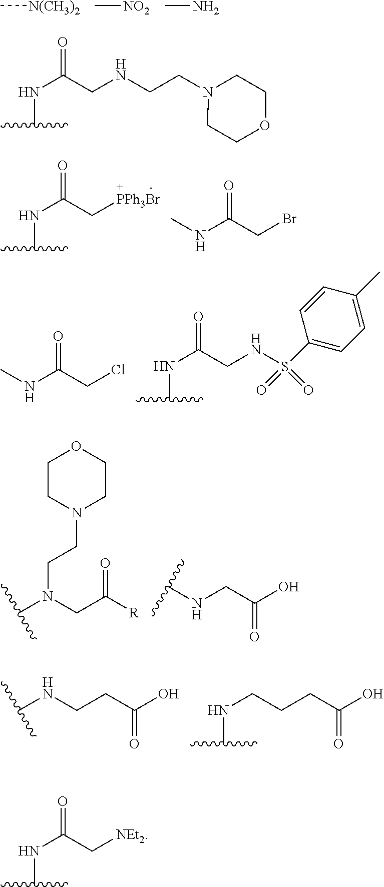

23. The pH-activable fluorescent probe according to claim 20, wherein R₂ and R₃ are methyl; R₄ is selected from the group consisting of: 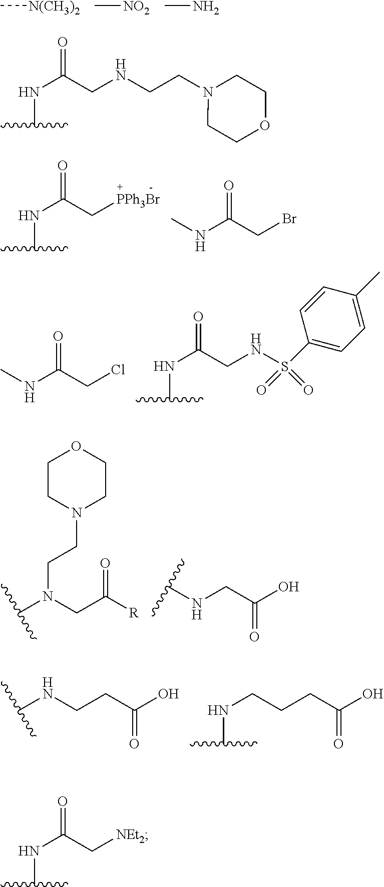
and R₅ is selected from the group consisting of:
—CO₂Et, —COOH, —CONHNH₂,

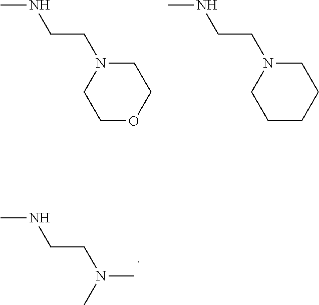

24. The pH-activable fluorescent probe according to claim 20, wherein R₅ is —CO₂Et; R₂ and R₃, independently, are selected from the group consisting of: 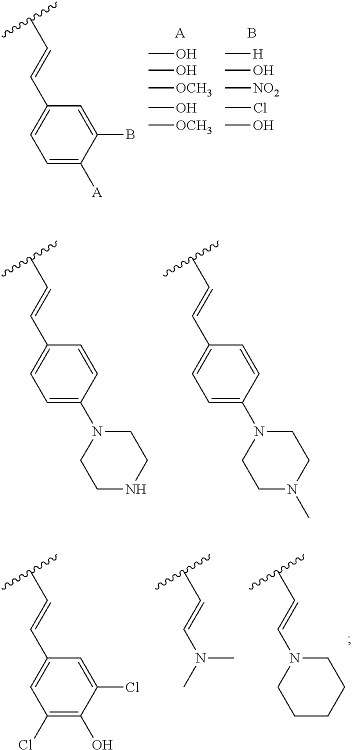
and R₄ is selected from the group consisting of:

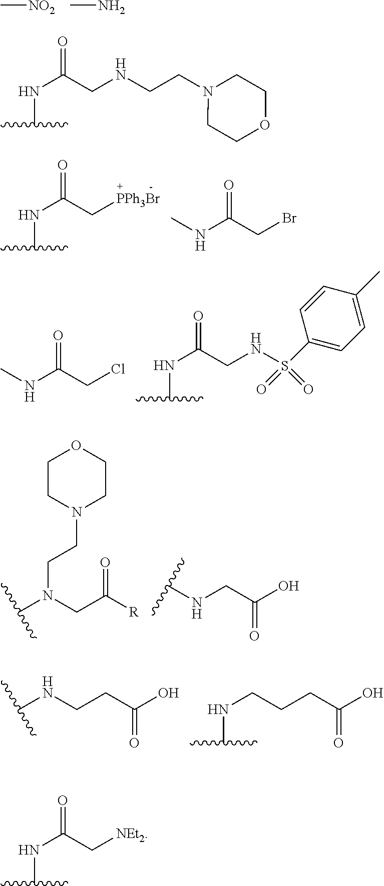

25. The pH-activable fluorescent probe according to claim 20, wherein said probe is useful for selective targeting lysosomal, mitochondrial, or nucleus.
